(19) United States
(12) Patent Application Publication
(10) Pub. No.: US 2025/0144619 A1
(43) Pub. Date: May 8, 2025

(54) Dermal Patch with a Diagnostic Test Strip

(71) Applicant: Satio, Inc., Boston, MA (US)

(72) Inventors: Namal NAWANA, Weston, MA (US); Ziad Tarik AL-SHAMSIE, San Diego, CA (US)

(21) Appl. No.: 19/013,669

(22) Filed: Jan. 8, 2025

Related U.S. Application Data
(62) Division of application No. 18/090,063, filed on Dec. 28, 2022, now Pat. No. 12,214,346.
Application No. 18/090,063 is a continuation-in-part of application No. 17/994,454, filed on Nov. 28, 2022, now Pat. No. 11,877,848.
Application No. 17/994,454 is a continuation of application No. 17/521,466, filed on Nov. 8, 2021, now Pat. No. 11,510,602.
Application No. 18/090,063 is a continuation-in-part of application No. 17/991,284, filed on Nov. 21, 2022, now Pat. No. 12,048,543.
Application No. 17/991,284 is a continuation-in-part of application No. 17/971,142, filed on Oct. 21, 2022, now Pat. No. 12,053,284.
Application No. 17/971,142 is a continuation-in-part of application No. 17/521,466, filed on Nov. 8, 2021, now Pat. No. 11,510,602.
Application No. 18/090,063 is a continuation-in-part of application No. 17/903,802, filed on Sep. 6, 2022.
Application No. 17/903,802 is a continuation of application No. 17/412,213, filed on Aug. 25, 2021, now Pat. No. 11,452,474.
Application No. 18/090,063 is a continuation-in-part of application No. 17/500,873, filed on Oct. 13, 2021, now Pat. No. 11,964,121.
(60) Provisional application No. 63/190,700, filed on May 19, 2021.
(60) Provisional application No. 63/174,956, filed on Apr. 14, 2021.

(51) Int. Cl. B01L 3/00; A61B 5/151

(52) U.S. Cl. CPC B01L 3/5023; A61B 5/15109; B01L 2200/04; B01L 2200/10; B01L 2200/16; B01L 2300/023; B01L 2300/044; B01L 2300/0672; B01L 2300/069; B01L 2300/0825; B01L 2400/049

(57) ABSTRACT

A system for analyzing a physiological sample includes a cartridge configured to attach to the skin of a subject. The cartridge includes: a processing fluid pack that is configured to release a processing fluid stored therein, a diagnostic test strip, and a vacuum pin. The system also includes a lancet with a needle. The lancet is configured to deploy the needle upon engagement with the cartridge to draw a physiological sample from the subject. The vacuum pin is configured to create a vacuum within the cartridge to draw the released processing fluid and the drawn physiological sample to the diagnostic test strip.

### RELATED APPLICATIONS

The present application is a divisional of U.S. patent application Ser. No. 18/090,063 filed on Dec. 28, 2022, which is a continuation-in-part of: (1) U.S. patent application Ser. No. 17/903,802 filed on Sep. 6, 2022, which is a continuation of U.S. application Ser. No. 17/412,213 filed on Aug. 25, 2021 (issued as U.S. Pat. No. 11,452,474), which claims the benefit of U.S. Provisional Application Nos. 63/190,700 and 63/174,956, filed on May 19, 2021 and Apr. 14, 2021, respectively; (2) U.S. patent application Ser. No. 17/500,873 filed on Oct. 13, 2021 (issued as U.S. Pat. No. 11,964,121); (3) U.S. patent application Ser. No. 17/994,454 filed on Nov. 28, 2022 (issued as U.S. patent Ser. No. 11/877,848), which is a continuation of U.S. patent application Ser. No. 17/521,466 filed on Nov. 8, 2021 (issued as U.S. Pat. No. 11,510,602); and (4) U.S. patent application Ser. No. 17/991,284 filed on Nov. 21, 2022 (issued as U.S. Pat. No. 12,048,543), which is a continuation-in-part of U.S. patent application Ser. No. 17/971,142 filed on Oct. 21, 2022 (now U.S. Pat. No. 12,053,284), which is a continuation-in-part of U.S. application Ser. No. 17/521,466 filed on Nov. 8, 2021 (issued as U.S. Pat. No. 11,510,602). Each of these applications is herein incorporated by reference in its entirety.

### TECHNICAL FIELD

The present teachings are generally directed to dermal patches that can be employed to detect a biomarker in a drawn physiological sample.

### BACKGROUND

Biomarkers are increasingly employed for diagnosis of various disease conditions as well as for assessing treatment protocols. In many cases, it is important to monitor the level of the biomarker over time (e.g., to assess the progression of a disease). The temporal monitoring of biomarkers via conventional techniques includes drawing a physiological fluid sample from a subject. These techniques may be cumbersome and painful to the subject. For example, the invasive nature of drawing a blood sample form a subject can cause discomfort and may lead to less cooperation from a subject, especially children, rendering multiple measurements of a target analyte (e.g., a biomarker) difficult.

Conventional devices allow for continuous monitoring of a target analyte (e.g., glucose monitors) typically suffer from several shortcomings, such as low sensitivity and/or specificity. Therefore, there is still a need for dermal patches for the detection a target analyte.

### SUMMARY

Aspects of the present disclosure address the above-referenced problems and/or others.

In one aspect, a system for analyzing a physiological sample includes a cartridge configured to attach to the skin of a subject. The cartridge includes a processing fluid pack that is configured to release a processing fluid stored therein, a diagnostic test strip, and a vacuum pin. The system further includes a lancet with a needle, wherein the lancet is configured to deploy the needle upon engagement with the cartridge to draw a physiological sample from the subject. The vacuum pin is configured to create a vacuum within the cartridge to draw the released processing fluid and the drawn physiological sample to the diagnostic test strip. In some embodiments, the drawn physiological sample and the released processing fluid interact to form a processed physiological sample and the diagnostic test strip is configured to detect a biomarker within the processed physiological sample. In other embodiments, the vacuum pin is moveable between an undeployed position and a deployed position at which the vacuum pin creates the vacuum within the dermal patch when in the deployed position. In some embodiments, the strength of the vacuum generated via movement of the pin is related, e.g., it is proportional on, a distance travelled by the vacuum pin.

In some embodiments, the cartridge further includes a vacuum chamber, wherein the vacuum pin is moveable between the undeployed position and the deployed position within the vacuum chamber. In other embodiments, the cartridge further includes a physiological sample channel in open communication with the vacuum chamber, wherein the physiological sample channel is configured to carry the drawn physiological sample, and wherein the vacuum draws the physiological sample to the diagnostic test strip via the physiological sample channel. In other embodiments, the cartridge further includes a moveable button that is configured to compress the processing fluid pack. In some embodiments, the button is configured to move from a locked position to a deployed position, and the cartridge is configured to prevent the button from compressing the processing fluid pack when the button is in the locked position and is configured to allow the button to compress the processing fluid pack when the button is in the deployed position. In other embodiments, the cartridge further includes a piercing element, and the button is configured to compress the processing fluid pack into the piercing element to rupture the processing fluid pack, thereby releasing a processing fluid contained in the fluid pack.

In certain embodiments, the lancet is configured to automatically deploy the needle upon engagement with the cartridge. In other embodiments, the lancet is configured to automatically retract the needle into a housing of the lancet after deployment. In certain embodiments, the system further comprises a computer system configured to image the diagnostic test strip and determine a result of a test associated with the diagnostic test strip based on the image. In other embodiments, the system also includes an electronic medical record database that stores a plurality of electronic medical records, the cartridge further includes a quick response code, and the computer system is configured to associate the quick response code with an electronic medical record within the electronic medical record database. In other embodiments, the computer system is configured to update the associated electronic medical record with the determined result. In certain embodiments, the processing fluid stored in the processing fluid pack includes a lysing agent. In other embodiments the processing fluid stored in the processing fluid pack includes other reagents (e.g., detergents, surfactants, etc.). In other embodiments, the processing fluid pack includes a buffer, including without limitation, TBS-T, Tris Buffered Saline-Tween, and TENT, and Tris Buffered saline with EDTA. In other embodiments, the processing fluid can be a buffer solution to neutralize pH. In other embodiments, the diagnostic test strip is a lateral flow test strip.

In another aspect, a method for analyzing a physiological sample, affixing a cartridge to the skin of a subject, inserting a lancet with a needle into the cartridge, wherein inserting the lancet into the cartridge deploys the needle to draw a physiological sample from the subject, moving a vacuum pin to create a vacuum within the cartridge which draws the drawn physiological sample to the diagnostic test strip, and rupturing a processing fluid pack of the cartridge to release a processing fluid to the diagnostic test strip. In certain embodiments, the method further includes moving a button of the cartridge to a deployed position to compress the processing fluid pack into a piercing element of the cartridge thereby rupturing the processing fluid pack. In other embodiments, inserting the lancet into the cartridge causes the lancet to automatically deploy the needle.

In yet another aspect, a cartridge configured to attach to skin of a subject includes a processing fluid pack that is configured to release a processing fluid stored therein, a diagnostic test strip, a vacuum pin, and a lancet with a needle. The lancet is configured to deploy the needle upon engagement with the cartridge to draw a physiological sample from the subject. The vacuum pin is configured to create a vacuum within the cartridge to draw the released processing fluid and the drawn physiological sample to the diagnostic test strip.

### BRIEF DESCRIPTION OF THE DRAWINGS

Aspects of the present disclosure may take form in various components and arrangements of components, and in various steps and arrangements of steps. The drawings are only for illustration purpose of preferred embodiments of the present disclosure and are not to be considered as limiting.

Features of embodiments of the present disclosure will be more readily understood from the following detailed description take in conjunction with the accompanying drawings in which:

FIGS. 1A and 1B depict a dermal patch system in accordance with an exemplary embodiment of the present disclosure;

FIGS. 2A and 2B-7A and 7B depict a cartridge of the dermal patch system in accordance with an exemplary embodiment of the present disclosure;

FIGS. 8A and 8B depict a base of the cartridge that includes a processing fluid pack and a diagnostic test strip in accordance with an exemplary embodiment of the present disclosure;

FIGS. 9 and 10 depict a lancet of the dermal patch system in accordance with an exemplary embodiment of the present disclosure

FIGS. 11A and 11B depict a housing of the lancet in accordance with an exemplary embodiment of the present disclosure;

FIGS. 12A and 12B depict a cap of the lancet in accordance with an exemplary embodiment of the present disclosure;

FIG. 13 depicts an inner sleeve of the lancet in accordance with an exemplary embodiment of the present disclosure;

FIG. 14 depicts a needle frame in accordance with an exemplary embodiment of the present disclosure;

FIGS. 15A and 15B-22A and 22B depict a cover of the cartridge of the dermal patch system in accordance with an exemplary embodiment of the present disclosure;

FIGS. 23A and 23B-40A and 40B depict a base of the dermal patch system in accordance with an exemplary embodiment of the present disclosure;

FIG. 41 depicts a lancet receiving element of the base of the dermal patch system in accordance with an exemplary embodiment of the present disclosure;

FIGS. 42A and 42B depict a vacuum pin of the dermal patch system in accordance with an exemplary embodiment of the present disclosure;

FIGS. 43A and 43B-45 depict a button of the dermal patch system in accordance with an exemplary embodiment of the present disclosure;

FIGS. 46A and 46B depict a test strip support with a diagnostic test stirp in accordance with an exemplary embodiment of the present disclosure;

FIGS. 47A, 47B, 48A, and 48B depict a test strip support in accordance with an exemplary embodiment of the present disclosure;

FIG. 49 depicts the lancet of the dermal patch system in an undeployed position in accordance with an exemplary embodiment of the present disclosure;

FIG. 50 depicts the lancet of the dermal patch system in a deployed position in accordance with an exemplary embodiment of the present disclosure;

FIG. 51 depicts the lancet of the dermal patch system in a retracted position in accordance with an exemplary embodiment of the present disclosure;

FIG. 52 depicts the lancet coupled to the cartridge, wherein the lancet is in a deployed position in accordance with an exemplary embodiment of the present disclosure;

FIG. 53 depicts the lancet coupled to the cartridge, wherein the lancet is in a retracted position in accordance with an exemplary embodiment of the present disclosure;

FIG. 54 depicts the lancet coupled to the cartridge, wherein a vacuum pin of the cartridge is in a deployed position in accordance with an exemplary embodiment of the present disclosure;

FIG. 55 depicts the lancet coupled to the cartridge, wherein a button and a vacuum pin of the cartridge are in a deployed position in accordance with an exemplary embodiment of the present disclosure;

FIG. 56 diagrammatically depicts an electronic medical record database, a computer system, and a dermal patch system with a quick response (“QR”) code in accordance with an exemplary embodiment of the present disclosure;

FIG. 57 diagrammatically depicts a computer system in accordance with an exemplary embodiment of the present disclosure;

FIG. 58 diagrammatically depicts a cloud computing environment in accordance with an exemplary embodiment of the present disclosure; and

FIG. 59 is a flow chart of a method for running a diagnostic test on a drawn physiological sample in accordance with an exemplary embodiment of the present disclosure.

### DETAILED DESCRIPTION

The present disclosure generally relates to a dermal patch that may be utilized to detect a biomarker in a physiological sample.

In some embodiments, a dermal patch may be used to perform a diagnostic test by detecting an analyte in a physiological sample. Dermal patches disclosed herein may allow a user to perform a diagnostic test in a variety of environments (e.g., in the home, in the field, in a medical facility, etc.).

Various terms are used herein in accordance with their ordinary meanings in the art, unless otherwise indicated.

The term “about,” as used herein, denotes a deviation of at most 10% relative to a numerical value. For example, about 100 μm means in the range of 90 μm-110 μm.

The term “substantially,” as used herein, refers to a deviation, if any, of at most 10% from a complete state and/or condition.

The term “subject” as used herein refers to a human subject or an animal subject (i.e., chicken, pig, cattle, dog, cat, etc.).

The term “physiological sample,” as used herein, includes fluid drawn from a subject and includes, but is not limited to, blood and interstitial fluid.

The term “lancet,” as used herein refers broadly to an element that can be used to provide a passageway, or facilitate the production of a passageway, in the skin for drawing a physiological sample.

The term “transparent,” as used herein, indicates that light can substantially pass through an object (e.g., a window) to allow visualization of a material disposed behind the object. For example, in some embodiments, a transparent object allows the passageway of at least 70%, or at least 80%, or at least 90% of visible light therethrough.

The term “vacuum,” as used herein, refers to a pressure less than atmospheric pressure and more particularly to a pressure that can facilitate the movement of a fluid (e.g., a physiological sample) within a dermal patch.

The term “needle” as used herein, refers to a component with a pointed tip that is configured to pierce an outer surface of an element (e.g., skin of a subject) to provide a passageway.

The term “diagnostic test strip” refers to a band/piece/strip of paper or other material configured to determine the presence or absence of a biomarker in a physiological sample.

The term “biomarker” refers to a biological molecule that is an indicator of a biological state or condition.

The present disclosure generally relates to a device, which is herein also referred to as a dermal patch or a dermal patch system, for detecting a biomarker in a physiological sample (e.g., bodily fluids such as blood, interstitial fluids, etc.) from a subject. In some embodiments discussed below, such a dermal patch system can include a cartridge that can be affixed to a subject's skin (e.g., via an adhesive layer) and a separate lancet that can be engaged with the cartridge to puncture the skin, thereby providing a passageway for extracting the physiological sample. As discussed in more detail below, the lancet can include a housing in which at least one needle that is configured for puncturing the skin is disposed. The lancet can further include a mechanism that can be transitioned between at least two states. In one state (herein referred to as a locked state), the mechanism retains the needle within the lancet in an undeployed position when the lancet is not engaged with the cartridge. When the lancet is coupled to the cartridge, the mechanism transitions to a second state (herein referred to as a released state). In the released state, the mechanism allows the needle to be deployed for puncturing the skin. For example, in some embodiments, the mechanism can include an upper locking portion that can retain an upper spring that is coupled to a needle platform (to which a needle is mounted) in a compressed state, thereby preventing the needle from transitioning into a deployed position. Further, the mechanism can include an upper interference member that prevents the movement of the needle platform when the mechanism is in the locked state.

The engagement of the lancet with the cartridge results in an automatic transition of the mechanism from the locked state to the released state, which transitions the needle into a deployed position in which the needle extends beyond the lancet and the cartridge housing to puncture the subject's skin. In some embodiments, the engagement of the lancet with the cartridge causes the upper locking member to release the needle platform, which in turn allows the upper spring to decompress and thus push down the needle platform thereby deploying the needle. In some embodiments, the mechanism can further include a lower interference member that restricts the downward movement of the needle platform, when the needle platform is released. In this manner the extent of the penetration of the needle into the skin can be controlled. In certain embodiments, the mechanism can also include a lower locking member that retains a lower spring in a compressed state. The downward movement of the needle platform can cause the release of the lower locking member to allow the lower spring to decompress and exert a restoring force on the needle platform to cause the retraction of the needle into the lancet housing.

In this manner, the lancet remains safe before it is engaged with the cartridge as the lancet is not capable of deploying the needle when the lancet is not engaged with the cartridge. Furthermore, in this manner, the lancet remains safe after drawing a physiological sample as the needle automatically retracts back into the lancet after being deployed.

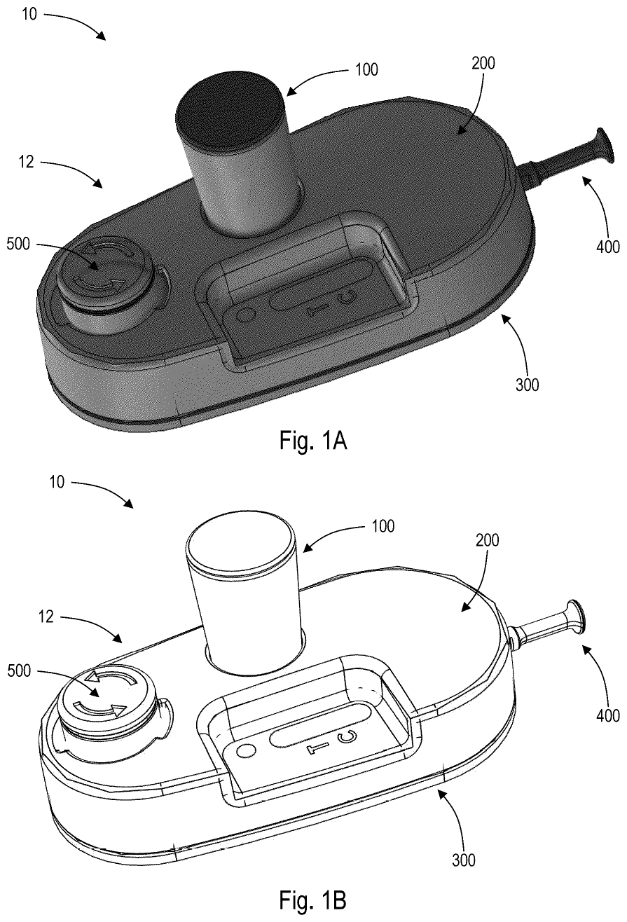

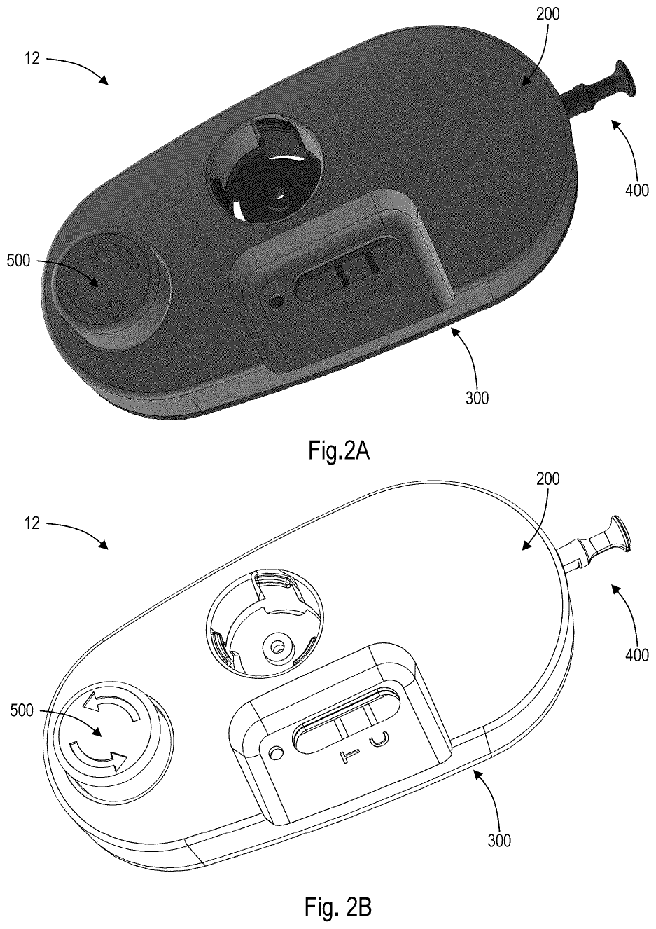

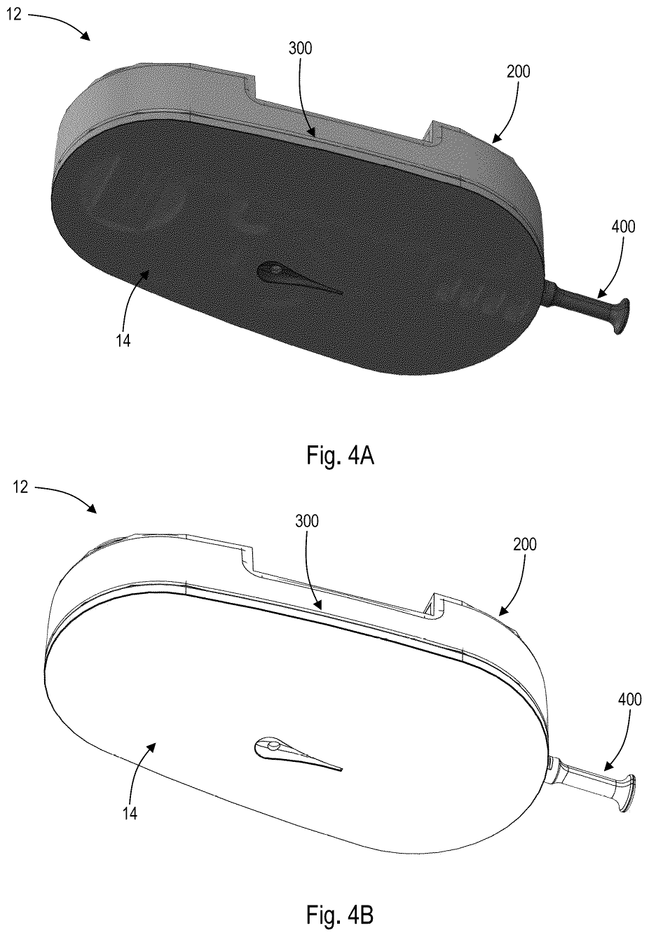

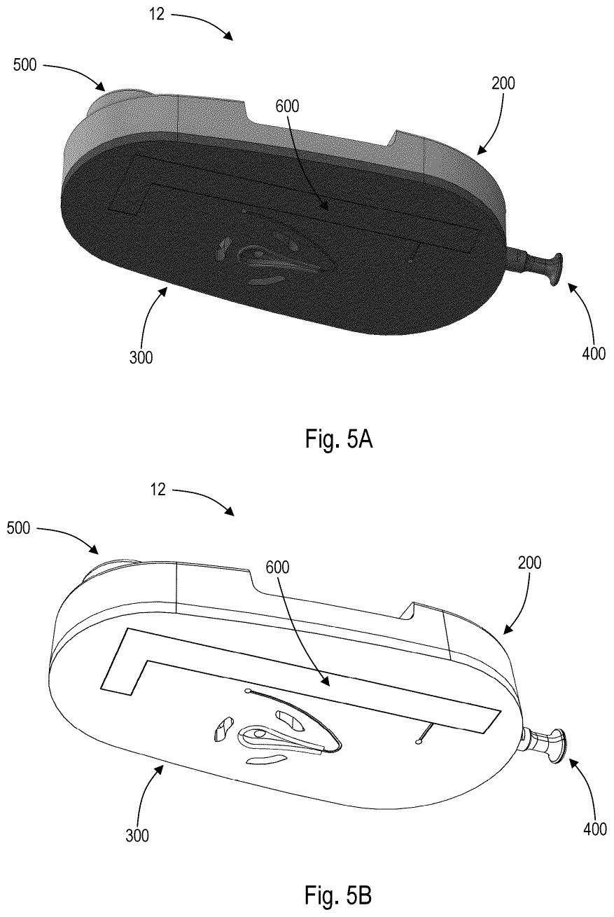

Referring now to FIGS. 1A and 1B, a dermal patch system 10 is shown in accordance with an exemplary embodiment. The dermal patch system 10 includes a cartridge 12 that can be affixed to a subject's skin via an adhesive layer 14. (FIGS. 4A and 4B). The dermal patch system 10 also includes a lancet 100 that can engage with the cartridge 12 to deploy a needle disposed within the lancet 100.

The dermal patch system 10 includes a lancet 100, a cartridge 12 that can be affixed to a subject's skin via an adhesive layer 14 (FIGS. 4A and 4B). As will be discussed in further detail herein, the lancet 100 can engage with the cartridge 12 to deploy a needle disposed within the lancet housing to puncture the subject's skin thereby drawing a physiological sample from the subject.

The cartridge 12 includes a cover 200 and a base 300 that can couple to the cover 200. For example, the cover 200 and the base 300 can be formed as two or more separate components that are removably coupled to one another (e.g., via a snap fitting). In other embodiments, the cover 200 and the base 300 form an integral unitary cartridge 12. In some of these embodiments, the cover 200 can be bonded or coupled to the base 300 via an adhesive, laser welding, heat sealing, heat activated adhesive, etc. The dermal patch system 10 also includes a vacuum pin 400. As will be discussed in further detail herein, the vacuum pin 400 can be disposed within the cartridge 12 and is configured to create a vacuum within the cartridge 12.

The cartridge 12 may be formed using a variety of suitable materials including, but not limited to, polymeric materials (e.g., polyolefins, polyethylene terephthalate (PET), polyurethanes, polynorbornenes, polyethers, polyacrylates, polyamides (Polyether block amide also referred to as Pebax®), polysiloxanes, polyether amides, polyether esters, trans-polyisoprenes, polymethyl methacrylates (PMMA), cross-linked trans-polyoctylenes, cross-linked polyethylenes, cross-linked polyisoprenes, cross-linked polycyclooctenes, inorganic-organic hybrid polymers, co-polymer blends with polyethylene and Kraton®, styrene-butadiene co-polymers, urethane-butadiene co-polymers, polycaprolactone or oligo caprolactone co-polymers, polylactic acid (PLLA) or polylactide (PL/DLA) co-polymers, PLLA-polyglycolic acid (PGA) co-polymers, photocross linkable polymers, etc.). In some embodiments, some of the cover 200 may be formed of poly(dimethylsiloxane) (PDMS) to allow visibility of components disposed within the cartridge 12.

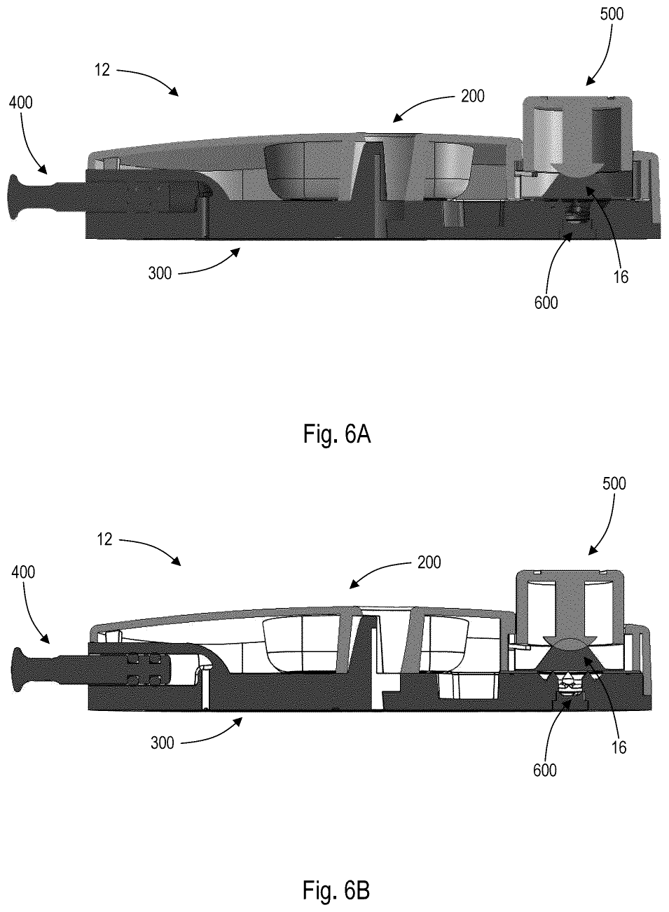

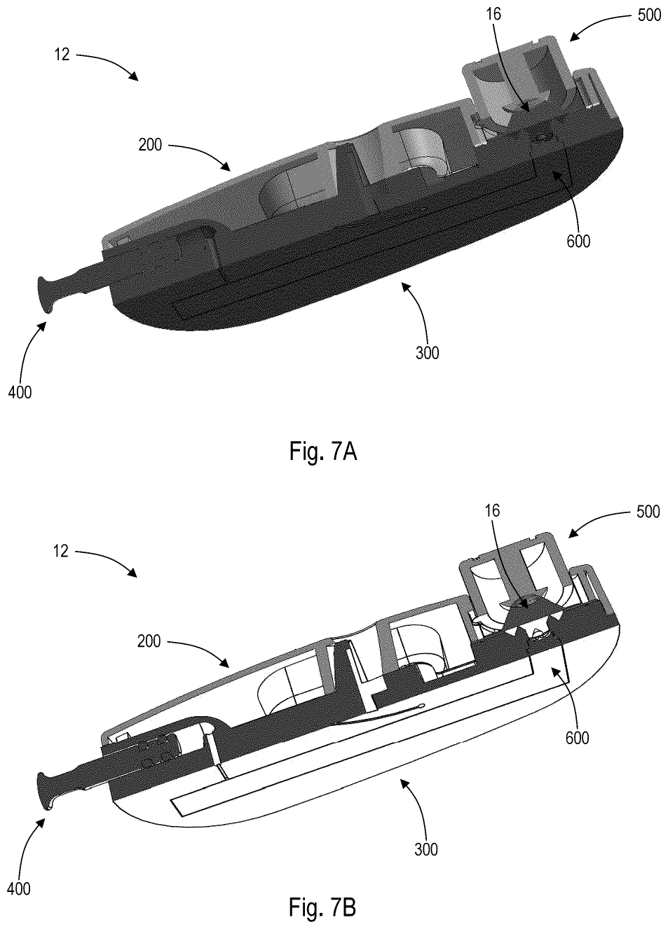

With particular reference to FIGS. 6A and 6B, the cartridge 12 further includes a sealed processing fluid pack 16 that is disposed in the base 300 of the cartridge 12 and a button 500 that extends through the cover 200. The processing fluid pack 16 is disposed within the cartridge 12 and contains a processing fluid (e.g., TBS-T, Tris Buffered Saline-Tween, and TENT, Tris Buffered saline with EDTA, etc.) for processing a physiological sample drawn form a subject. The processing fluid pack 16 is formed of a frangible membrane. As will be discussed in further detail herein, the button 500 is configured to apply a pressure upon the processing fluid pack 16 which causes the processing fluid pack 16 to rupture thereby releasing the processing fluid.

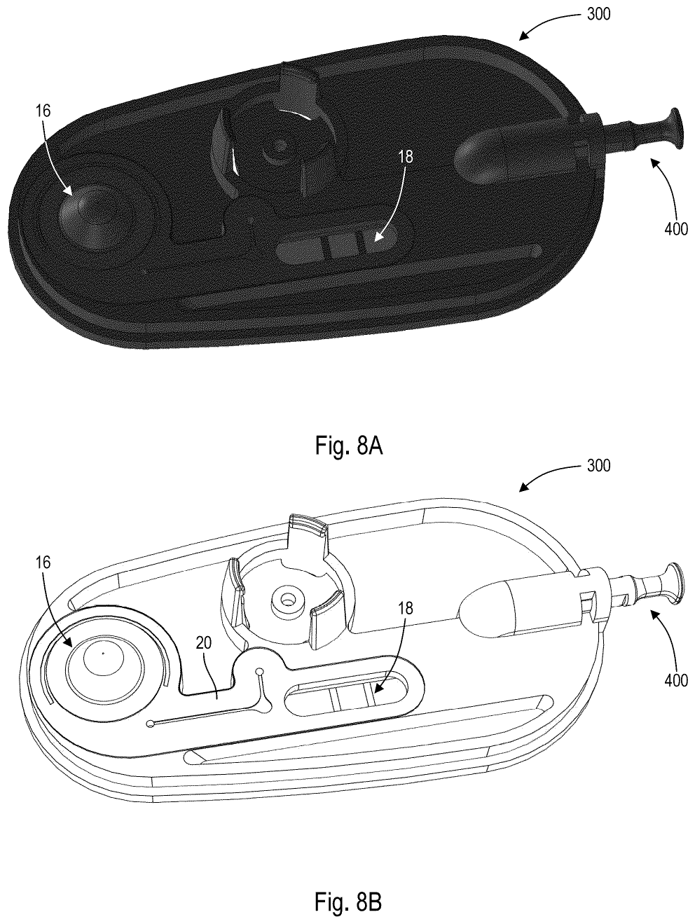

The cartridge 12 also includes a test strip support 600 that is configured to retain a diagnostic test strip 18. The test strip support 600 is configured to couple to the base 300 such that the diagnostic test strip 18 (FIG. 8B) is disposed within the cartridge 12 when the test strip support 600 is coupled to the base 300. The cartridge 12 further includes a film 20 (FIG. 8B) disposed on the base 300. As will be discussed in further detail herein, the film 20 seals at least a portion of the base 300.

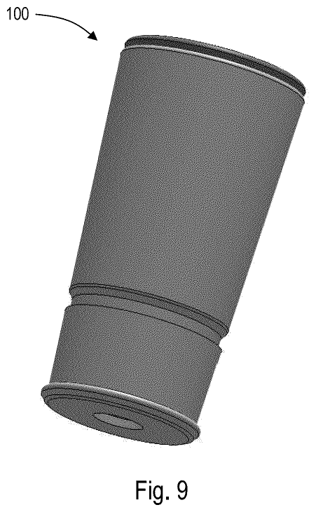

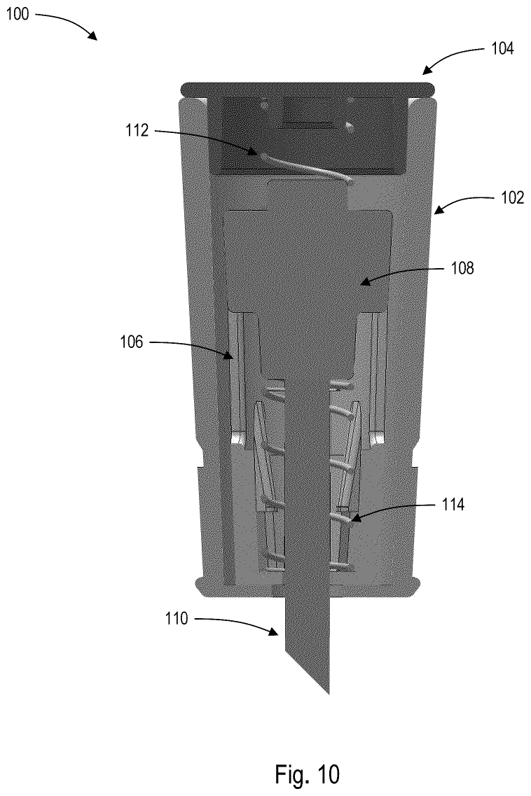

Referring now to FIGS. 9 and 10, the lancet 100 is shown in accordance with an exemplary embodiment. The lancet 100 includes a housing 102 in which various components of the lancet are disposed and a cap 104 that is coupled to the housing 102. The lancet 100 can further include an inner sleeve 106 within the housing 102 and a needle frame 108 that is disposed within the inner sleeve 106 and onto which a needle 110 is mounted. The lancet 100 also can include an injection spring 112 and a retraction spring 114 that move a needle of the lancet between various positions.

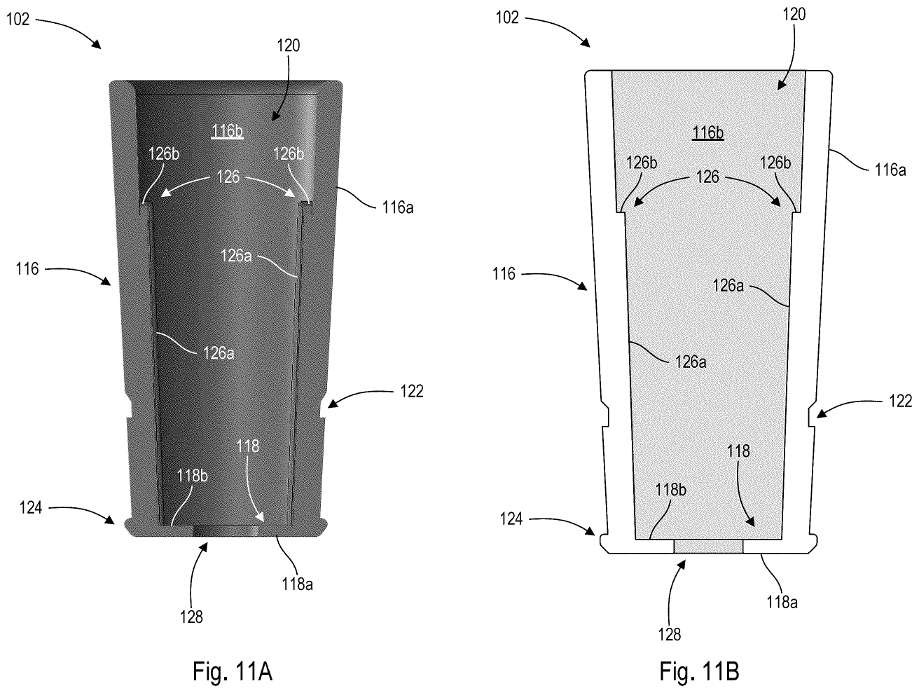

With particular reference to FIGS. 11A and 11B, the housing 102 includes a side wall 116 and a bottom wall 118. The side wall 116 includes an outer surface 116a and an opposed inner surface 116b. The bottom wall 118 includes an outer surface 118a and an opposed inner surface 118b. The side wall 116 extends vertically from the bottom wall 118. The side wall 116 has a generally cylindrical shape and the bottom wall 118 is generally circular in shape and is concentric relative to a longitudinal axis of the generally cylindrical side wall and covers a lower opening formed by the generally cylindrical side wall. The inner surface 116b of the side wall 116 and the inner surface 118b of the bottom wall 118 define an inner volume 120.

The outer surface 116a defines a notch 122 that extends circumferentially around the outer surface 116a of the side wall 116. As will be discussed in further detail herein, the notch 122 is shaped and dimensioned to couple to a locking member of the cartridge 12 via a snap fit. The housing 102 further includes a rim 124 that extends circumferentially around the outer surface 116a of the side wall 116. The inner surface 116b defines a first and second column 126 that extend vertically from the inner surface 118b of the bottom wall 118. The columns 126 includes an inner surface 126a and a top surface 126b. The inner surface 126a extends vertically between the inner surface 118b of the bottom wall 118 and the top surface 126b. The top surface 126b extends longitudinally between the inner surface 116b of the side wall 116 and the inner surface 126a.

As will be discussed in further detail herein, before the lancet 100 is inserted into the cartridge 12 the columns 126 retain the needle 110 of the lancet 100 in an undeployed position.

The bottom wall 118 defines an aperture 128 that extends through the bottom wall 118. Stated another way, the aperture 128 extends between the outer surface 118a and the inner surface 118b of the bottom wall 118. As will be discussed in further detail herein, when the lancet is activated via engagement with the cartridge 12, the needle of the lancet 100 is activated to extend through the aperture 128 and puncture the subject's skin thereby providing a passageway through which a physiological sample can be drawn from a subject.

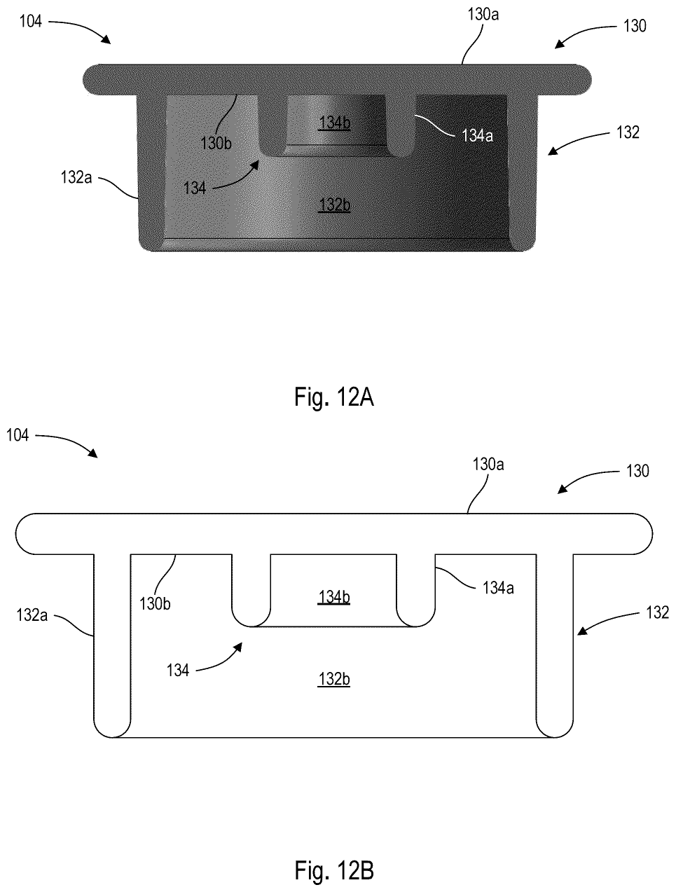

With particular reference to FIGS. 12A and 12B the cap 104 includes a top wall 130 with an outer surface 130a and an opposed inner surface 130b. The cap 104 also includes a side wall 132 with an outer surface 132a and an opposed inner surface 132b. The top wall 130 extends longitudinally from and perpendicular to the side wall 132. The side wall 132 extends vertically from and perpendicular to the top wall 130. The top wall 130 and the side wall 132 are generally circular in shape and are concentric with one another. The cap 104 also includes an inner cylinder 134 with an outer surface 134a and an opposed inner surface 134b. The inner cylinder 134 extends vertically from and perpendicular to the top wall 130. The inner cylinder 134 is concentric with the top wall 130 and the side wall 132.

When the cap 104 is coupled to the housing 102 the side wall 132 extends into the inner volume 120 of the housing 102 and at least a portion of the side wall 132 contacts the inner surface 116b of the side wall 116 such that the cap 104 couples to the housing 102 via an interference fit.

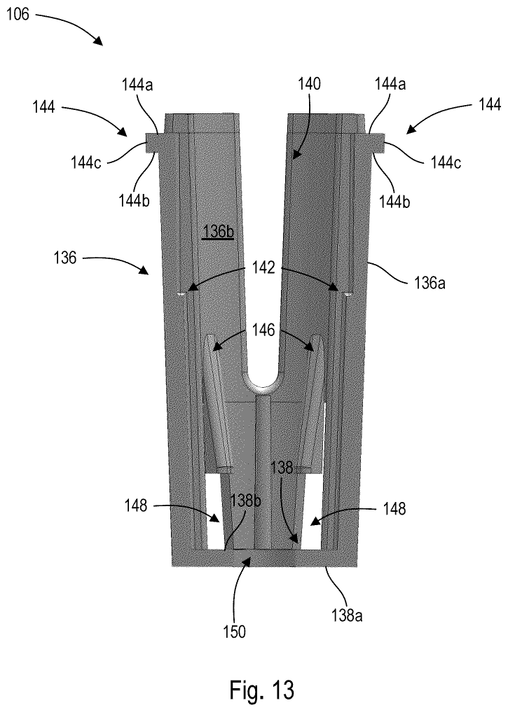

As depicted in FIG. 13, the inner sleeve 106 includes a side wall 136 and a bottom wall 138. The side wall 136 includes an outer surface 136a and an opposed inner surface 136b. The bottom wall 138 includes an outer surface 138a and an opposed inner surface 138b. The side wall 136 extends vertically from the bottom wall 138. The side wall 136 is substantially cylindrical and the bottom wall 138 are generally circular in shape and are concentric with one another. The inner surface 136b of the side wall 136 and the inner surface 138b of the bottom wall 138 define an inner volume 140. The inner surface 136b defines a plurality of columns 142 each of which extends vertically from and perpendicular to the inner surface 138b of the bottom wall 138. As will be discussed in further detail herein, when the needle frame 108 is in a deployed position, a portion of the needle frame 108 rests upon the columns 142.

The inner sleeve 106 further includes a plurality of ledges 144 that extend circumferentially about the side wall 136. Each ledge 144 includes a top surface 144a, an opposed bottom surface 144b and an outer surface 144c that extends between the top surface 144a and the bottom surface 144b. The inner sleeve 106 also includes a plurality of locking members 146 that extend from the inner surface 136b of the side wall 136. As will be discussed in further detail herein, the proximal end of the locking members 146 retains the retraction spring 114 in a compressed state in absence of engagement between the lancet 100 and the cartridge 12. The side wall 136 further defines a plurality of openings 148 that extend through the side wall 136. Stated another way, the openings 148 extend between the outer surface 136a and the inner surface 136b of the side wall 136. Each of the openings 148 are aligned with a proximal end of a locking member 146 to allow the proximal end of a locking member 146 to extend therethrough.

The bottom wall 138 defines an aperture 150 that extends through the bottom wall 138. Stated another way, the aperture 150 extends between the outer surface 138a and the inner surface 138b of the bottom wall 138. The aperture 150 is concentric with the aperture 128 of the housing 102. As will be discussed in further detail herein, when in a deployed position, the needle 110 of the lancet 100 extends through the aperture 150 of the inner sleeve 106 as well as the aperture 128 of the housing 102.

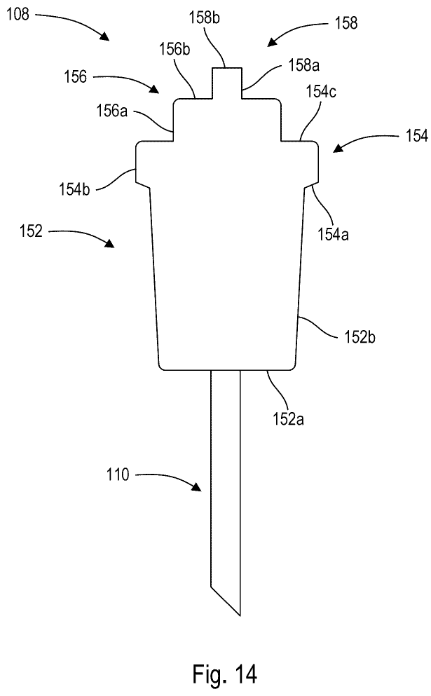

As depicted in FIG. 14, the needle frame 108 includes a first cylinder 152 and a second cylinder 154 disposed vertically above the second cylinder 154. The first cylinder 152 includes a bottom surface 152a and an outer surface 152b. The second cylinder 154 is disposed vertically above the first cylinder 152 and the third cylinder 156 is disposed vertically above the second cylinder 154. The first cylinder 152 includes a bottom surface 152a and an outer surface 152b and the second cylinder 154 includes a bottom surface 154a, an outer surface 154b and a top surface 154c. The third cylinder 156 includes an outer surface 156a and a top surface 156b. Similarly, the protrusion 158 includes an outer surface 158a and a top surface 158b.

The bottom surface 152a of the first cylinder 152 extends circumferentially about the outer surface 152b of the first cylinder. The outer surface 152b of the first cylinder 152 extends vertically between the bottom surface 152a of the first cylinder 152 and the bottom surface 154a of the second cylinder 154. The bottom surface 154a of the second cylinder 154 extends at an angle longitudinally between the outer surface 152b of the first cylinder and the outer surface 154b of the second cylinder 154. The outer surface 154b extends vertically between the bottom surface 154a and the top surface 154c of the second cylinder 154. The top surface 154c of the second cylinder 154 extends longitudinally between the outer surface 154b of the second cylinder and the outer surface 156a of the third cylinder 156. The outer surface 156a extends vertically between the top surface 154c of the second cylinder and the top surface 156b of the third cylinder. The top surface 156b of the third cylinder extends longitudinally between the outer surface 156a of the third cylinder 156 and the outer surface 158a of the protrusion 158. The outer surface 158a extends vertically between the top surface 156b of the third cylinder and the top surface 158b of the protrusion 158. The top surface 158b of the protrusion 158 extends across a proximal end of the outer surface 158a.

The injection spring 112 (FIG. 10) extends vertically between the cap 104 and the needle frame 108. More specifically, a distal end of the injection spring 112 contacts the inner surface 130b of the top wall 130 and a proximal end of the injection spring 112 contacts the top surface 154c of the second cylinder 154. The distal end of the injection spring 112 extends circumferentially around the outer surface 134a of the inner cylinder 134. The proximal end of the injection spring 112 extends circumferentially around the third cylinder 156 and around the protrusion 158.

The needle frame 108 supports the needle 110. In some embodiments, the needle 110 is molded into the first cylinder 152 or is attached to the bottom surface 152a of the first cylinder 152 (e.g., via an adhesive).

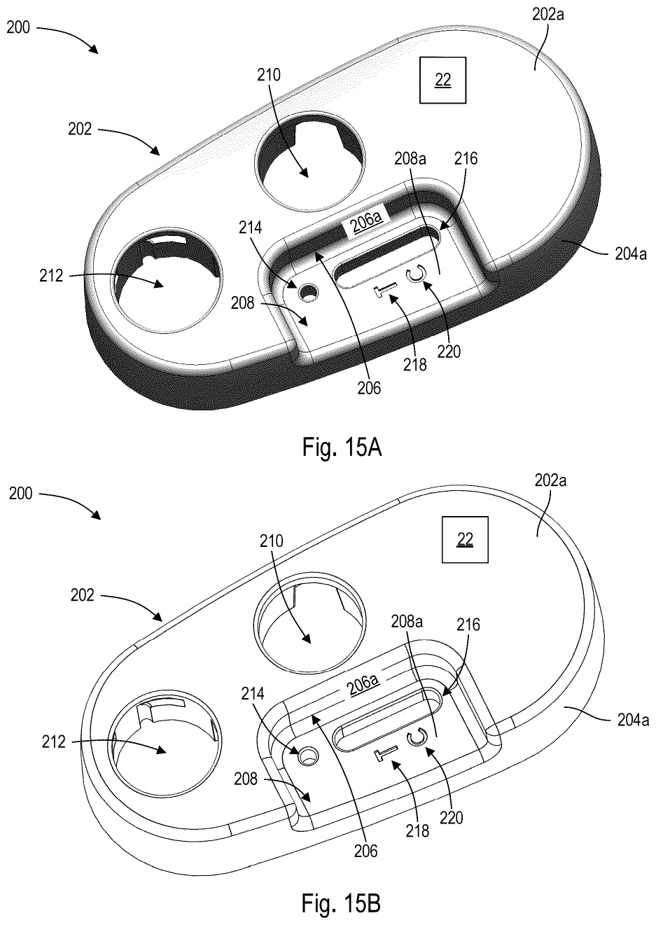

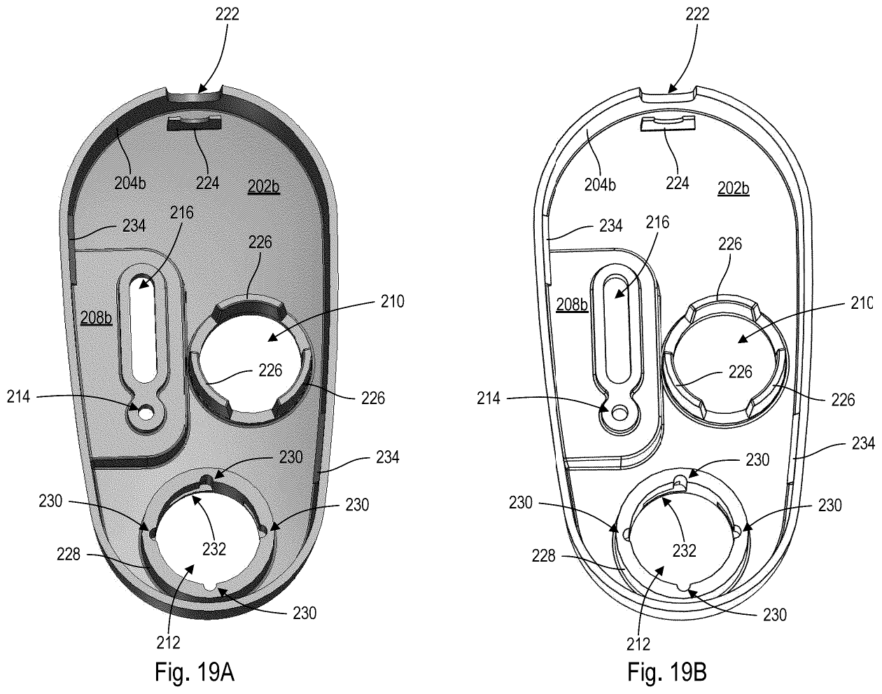

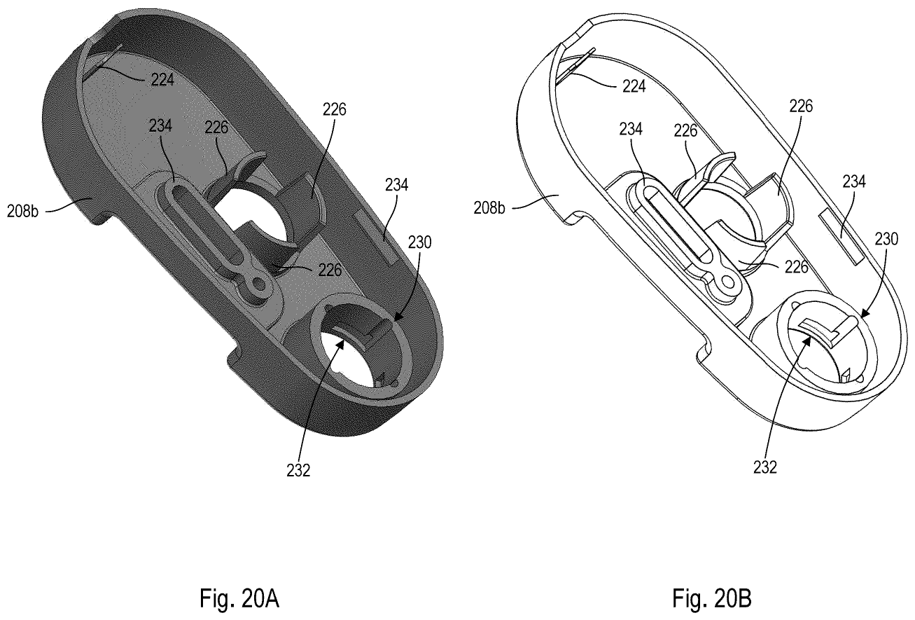

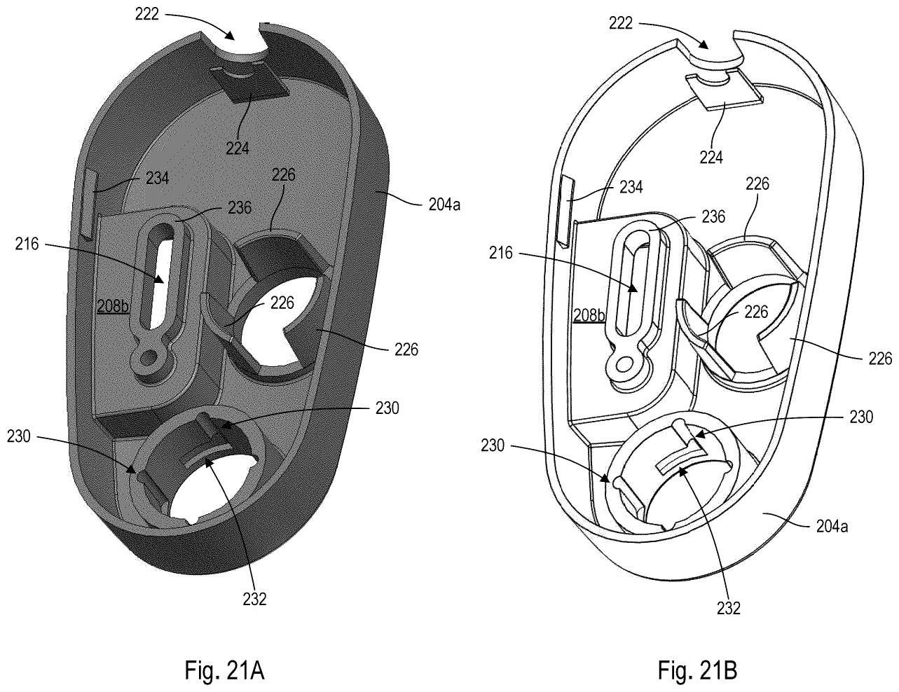

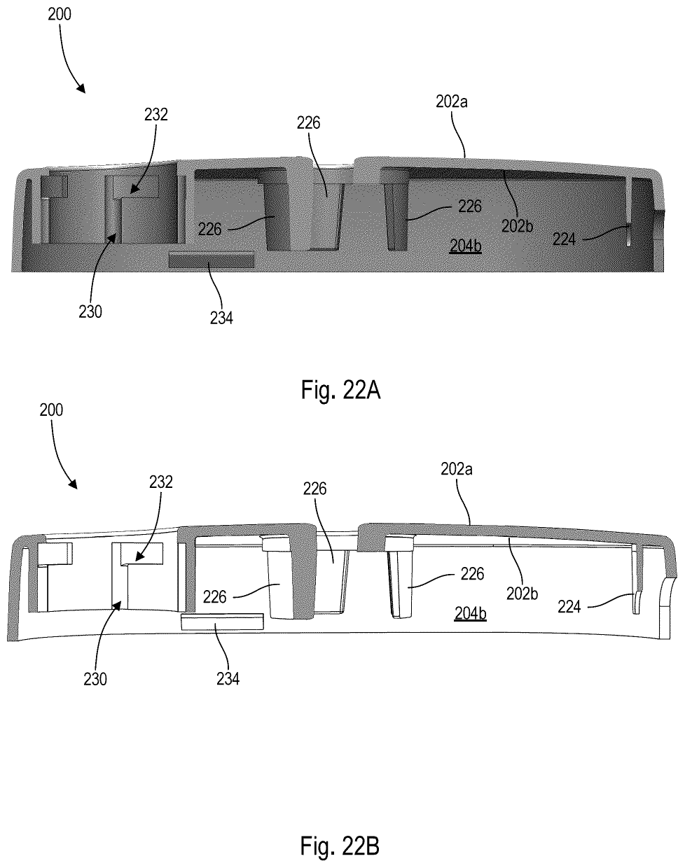

Referring now to FIGS. 15A and 15B-22A and 22B, the cover 200 is shown in accordance with an exemplary embodiment.

In this embodiment, the cover 200 includes a top wall 202 and a side wall 204. The side wall 204 extends vertically from and perpendicular to the top wall 202. The top wall 202 extends longitudinally from and perpendicular to the side wall 204. The top wall 202 includes an outer surface 202a and an opposed inner surface 202b. The side wall 204 includes an outer surface 204a and an opposed inner surface 204b.

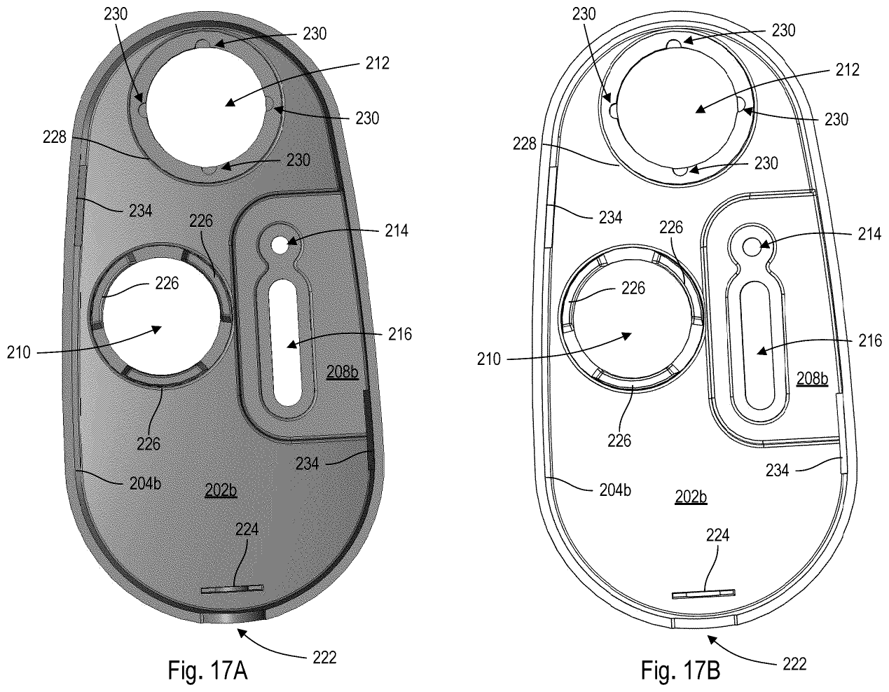

The cover 200 further includes a vertical wall 206 and a horizontal wall 208. The vertical wall 206 extends vertically between and perpendicular to the top wall 202 and the horizontal wall 208. The horizontal wall 208 extends longitudinally between and perpendicular to the side wall 204 and the vertical wall 206. The vertical wall 206 includes an outer surface 206a and an opposed inner surface 206b. The horizontal wall 208 includes an outer surface 208a and an opposed inner surface 208b (FIG. 17B).

The top wall 202 defines a lancet aperture 210 and a button aperture 212. The lancet aperture 210 and the button aperture 212 are generally circular in shape and extend through the top wall 202. Stated another way, the lancet aperture 210 and the button aperture 212 extend between the outer surface 202a and the inner surface 202b of the top wall 202. The lancet aperture 210 is shaped and dimensioned to accommodate at least a portion of the lancet 100. As will be discussed in further detail herein, the lancet aperture 210 allows the lancet 100 to couple to the base 300. That is, the lancet aperture 210 is shaped to accommodate the lancet 100 such that the lancet 100 can be engaged with the cartridge 12.

The horizontal wall 208 defines a sample viewing aperture 214 and a test strip viewing aperture 216. The sample viewing aperture 214 and the test strip viewing aperture 216 extend through the horizontal wall 208. Stated another way, the sample viewing aperture 214 and the test strip viewing aperture 216 extend between the outer surface 208a and the inner surface 208b. The sample viewing aperture 214 and the test strip viewing aperture 216 allow a user of the dermal patch system 10 to view components (e.g., the diagnostic test strip 18) disposed within the dermal patch system 10. In some embodiments, the cover 200 can further include a transparent window(s) (not shown), e.g., formed of PDMS, that extends across the sample viewing aperture 214 and the test strip viewing aperture 216.

The horizontal wall 208 further includes a test result indicator 218 and a control indicator 220 disposed on the outer surface 208a. As will be discussed in further detail herein, the test result indicator 218 and the control indicator 220 can be used to determine the presence or absence of a biomarker.

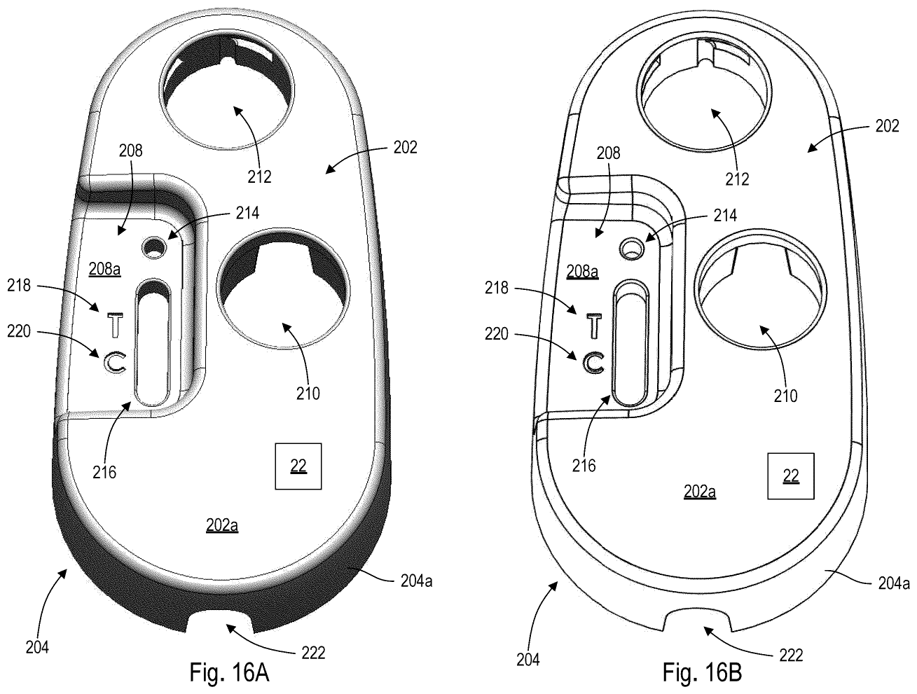

With particular reference to FIGS. 16A and 16B, the side wall 204 defines U-shaped opening 222, which extends through the side wall 204. Stated another way, the U-shaped opening 222 extends between the outer surface 204a and the inner surface 204b of the side wall 204. The U-shaped opening 222 is shaped and dimensioned to accommodate at least a portion of the vacuum pin 400. As will be discussed in further detail herein, U-shaped opening 222 allows the vacuum pin 400 to be received by a receptacle (which can be in the form of a channel) within the cartridge 12. That is, the U-shaped opening 222 is shaped to accommodate the vacuum pin 400 such that at least a portion of the vacuum pin 400 can extend through the side wall 204 to be disposed within a receptacle provided in the base 300.

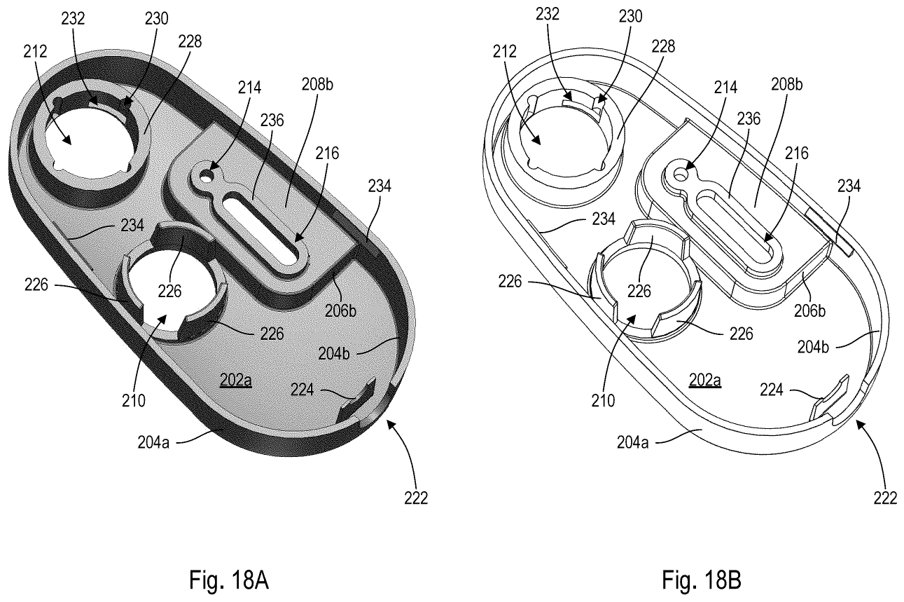

With reference to FIGS. 18A and 18B, the cover 200 further includes a U-shaped locking member 224 and a plurality of projection members 226. The U-shaped locking member 224 is aligned with the U-shaped opening 222. The U-shaped locking member 224 extends vertically from and perpendicular to the inner surface 202b of the top wall 202. When the cover 200 is coupled to the base 300, the U-shaped locking member 224 retains the vacuum pin 400 within the base 300 and allows the vacuum pin 400 to move a predetermined distance while remaining within the cartridge 12. The plurality of projection members 226 extend vertically from and perpendicular to the inner surface 202b of the top wall 202. The plurality of projection members 226 are disposed around the perimeter of the lancet aperture 210. As will be discussed in further detail herein, the plurality of projection members 226 help secure the cover 200 to the base 300.

The cover 200 also includes a button guide 228 that is generally circular in shape. The button guide 228 extends from and perpendicular to the inner surface 202b of the top wall 202. The button guide 228 extends circumferential around the button aperture 212.

The button guide 228 includes a plurality of vertical grooves 230 that extend vertically along an inner surface of the button guide 228 and a plurality of horizontal grooves 232 that extend horizontally along the inner surface of the button guide 228. Each vertical groove 230 is in open communication with a horizontal groove 232 and each vertical groove 230. Furthermore, each vertical groove 230 extends to a proximal end of the button guide 228.

The cover 200 further includes a plurality of locking members 234. The locking members 234 extend vertically from and perpendicular to the inner surface 204b of the side wall 204. As will be discussed in further detail herein, the locking members 234 couple the cover 200 to the base 300. The cover 200 also includes a support structure 236, which extends vertically from and perpendicular to the inner surface 208b of the horizontal wall 208. The support structure 236 extends around the viewing aperture 214 and the test strip viewing aperture 216 in such a manner that the support structure 236 does not prevent a user from viewing components disposed within the cartridge 12 when the cover 200 is coupled to the base 300.

In some embodiments, as depicted in FIGS. 15A, 15B, 16A, and 16B, the cover 200 can include a quick response (“QR”) code 22 disposed on the outer surface 202a of the top wall 202. As will be discussed in further detail herein, the QR code 22 can be associated with an electronic medical record (“EMR”) stored in an electronic medical record database and may aid in preserving a chain of custody of the cartridge 12.

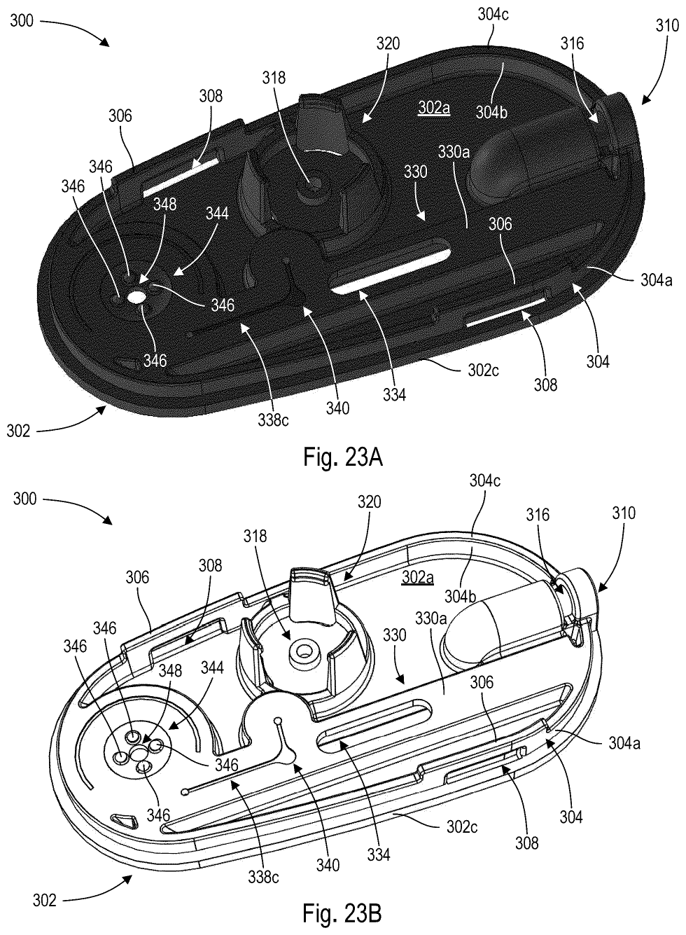

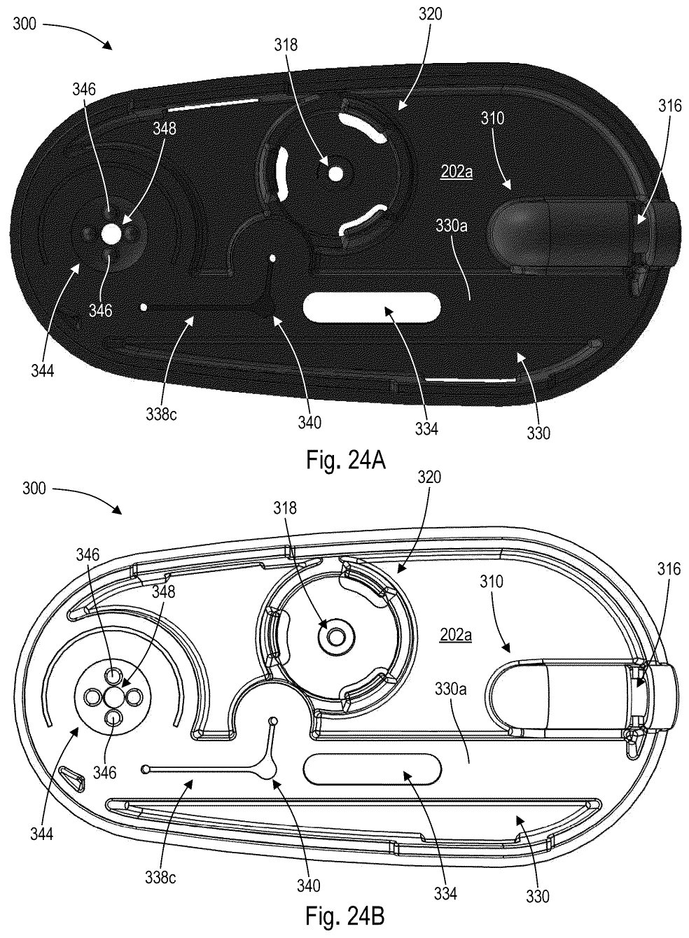

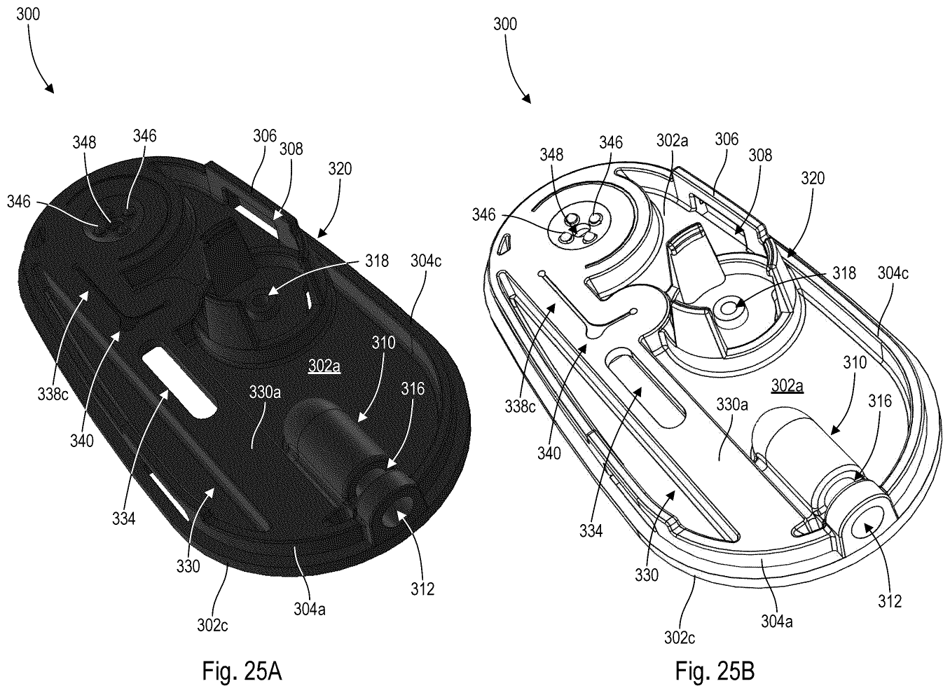

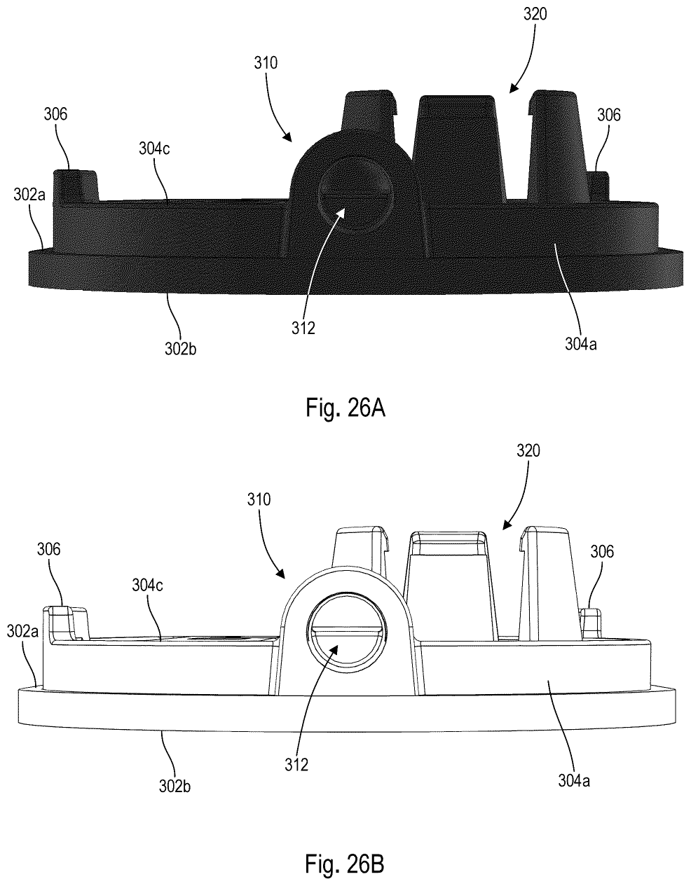

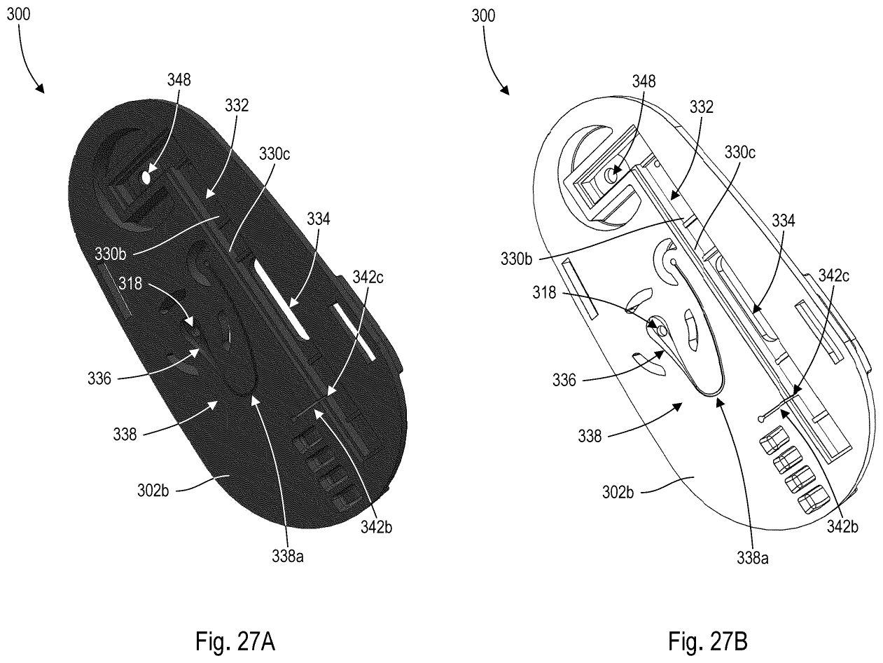

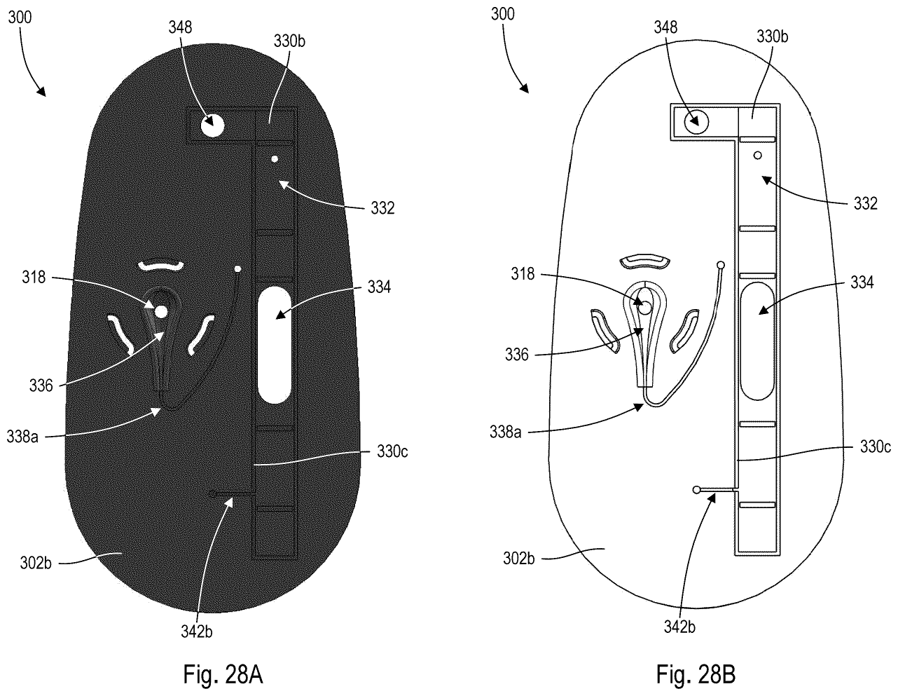

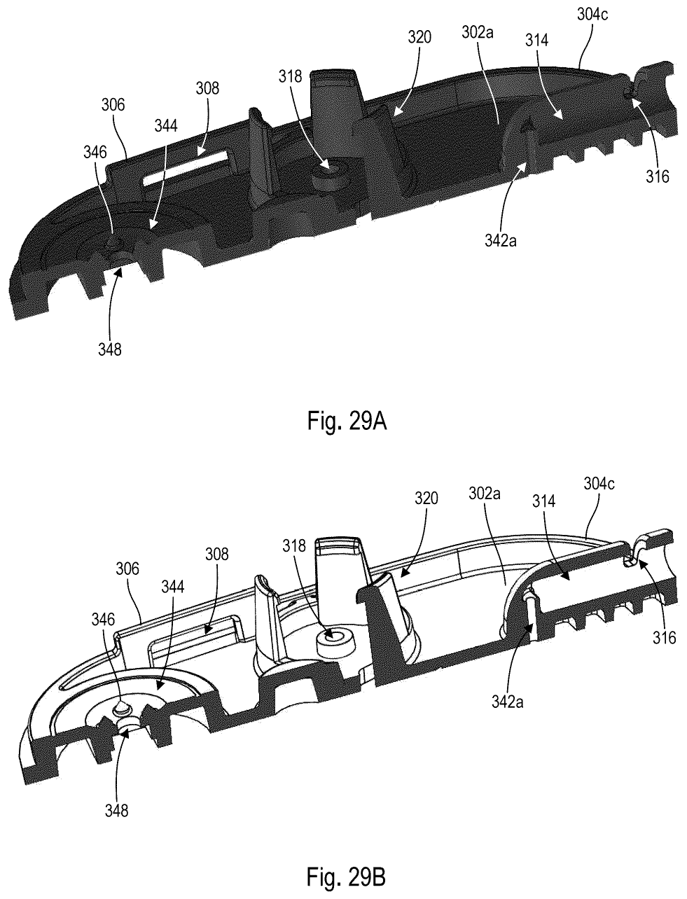

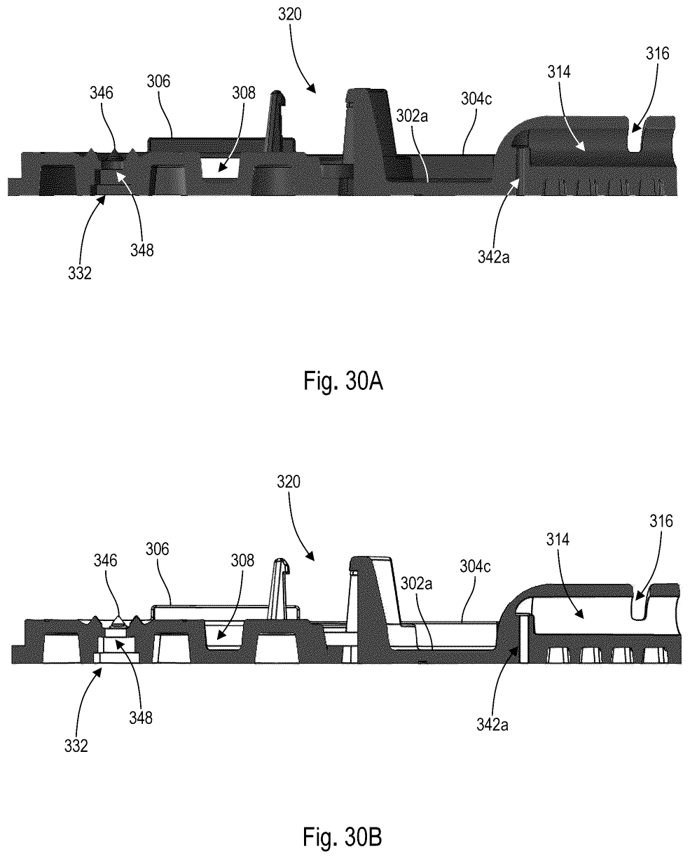

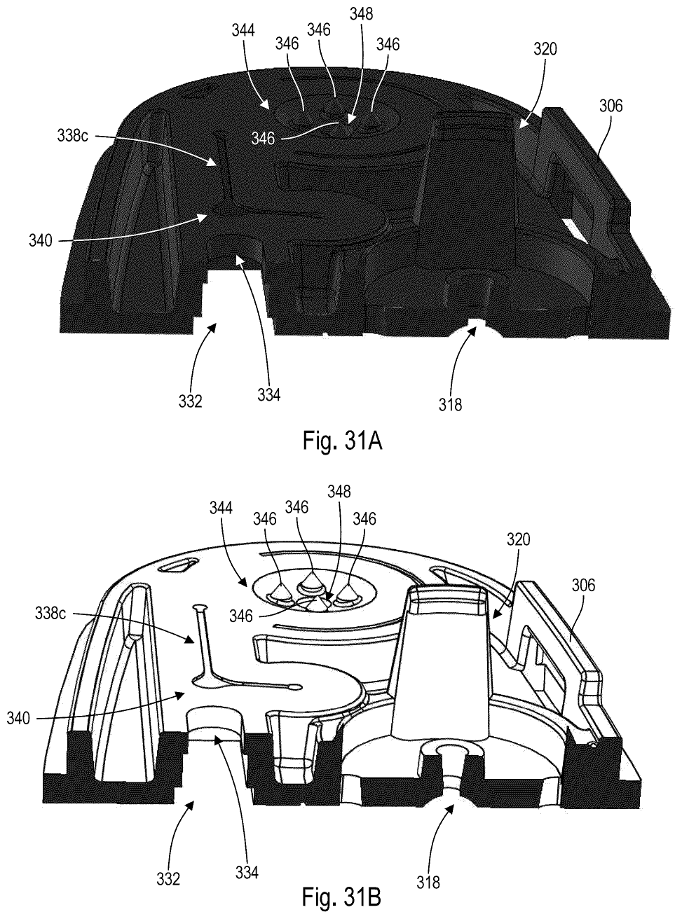

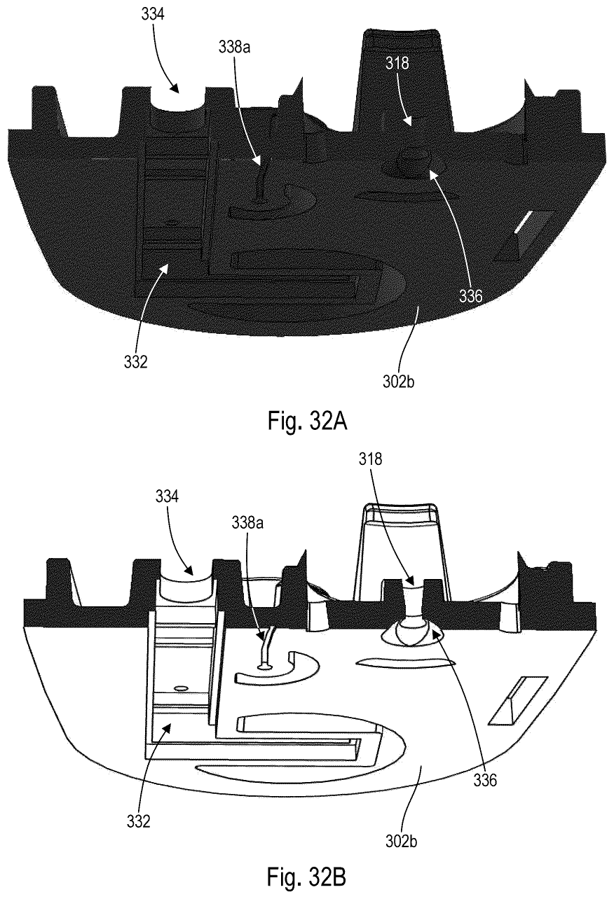

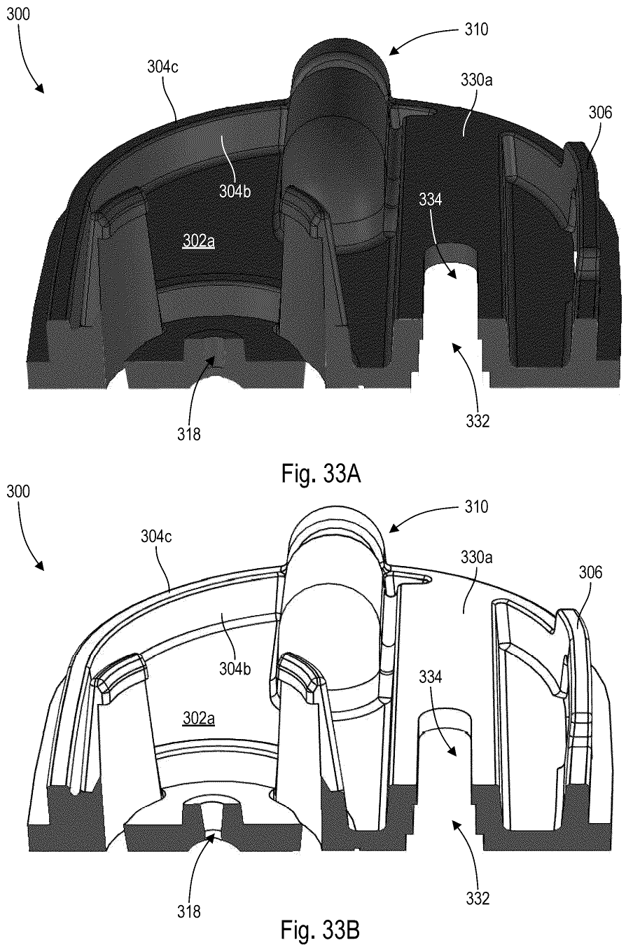

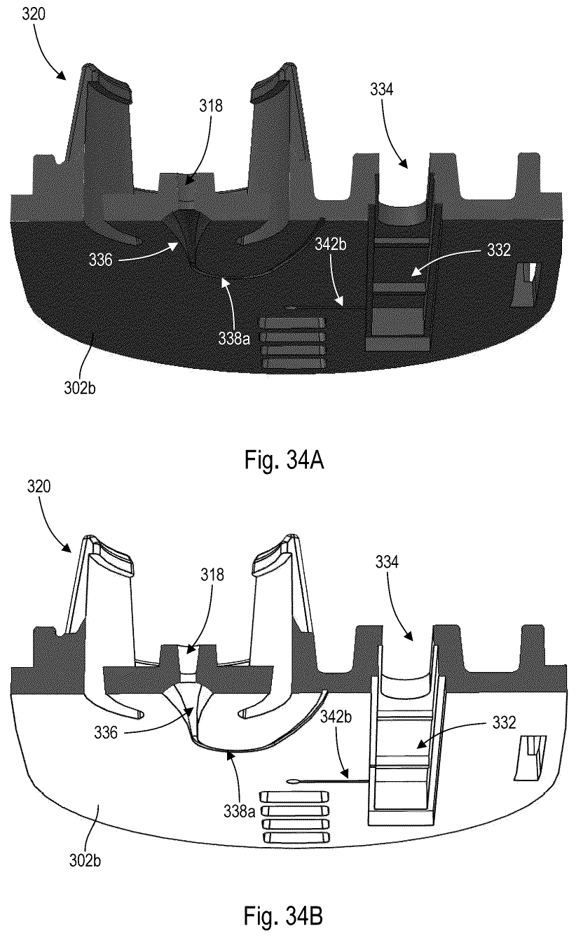

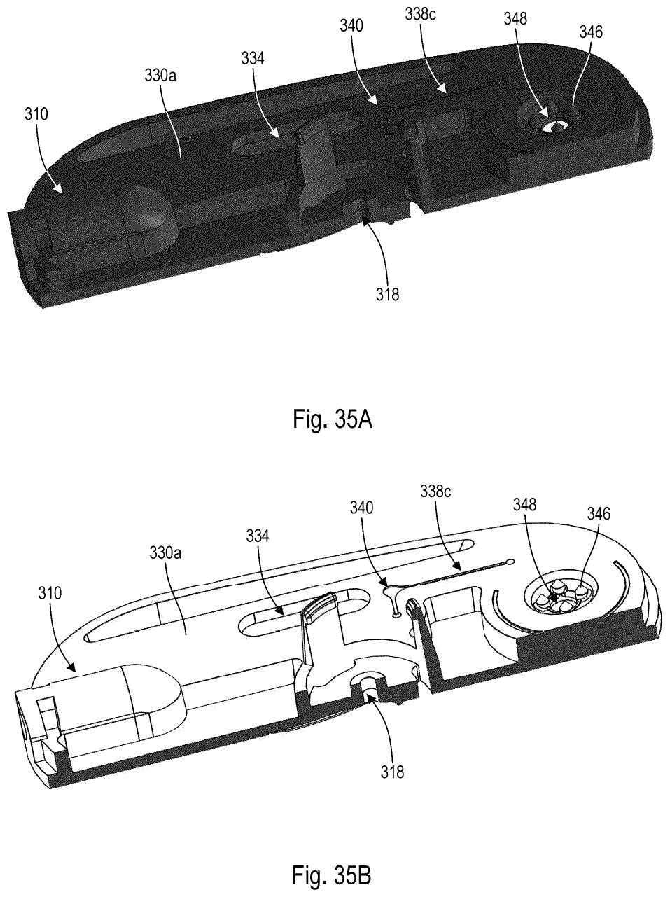

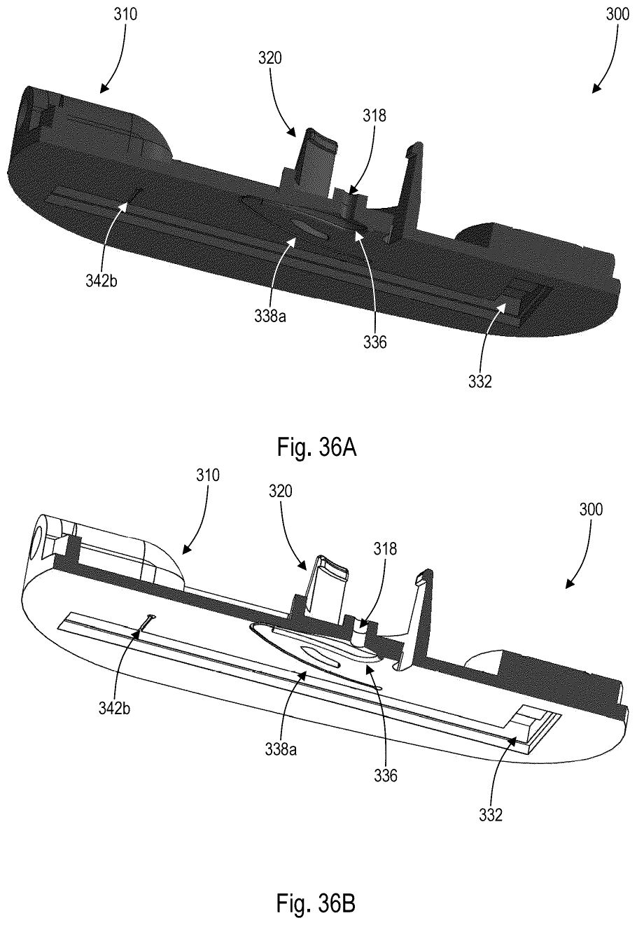

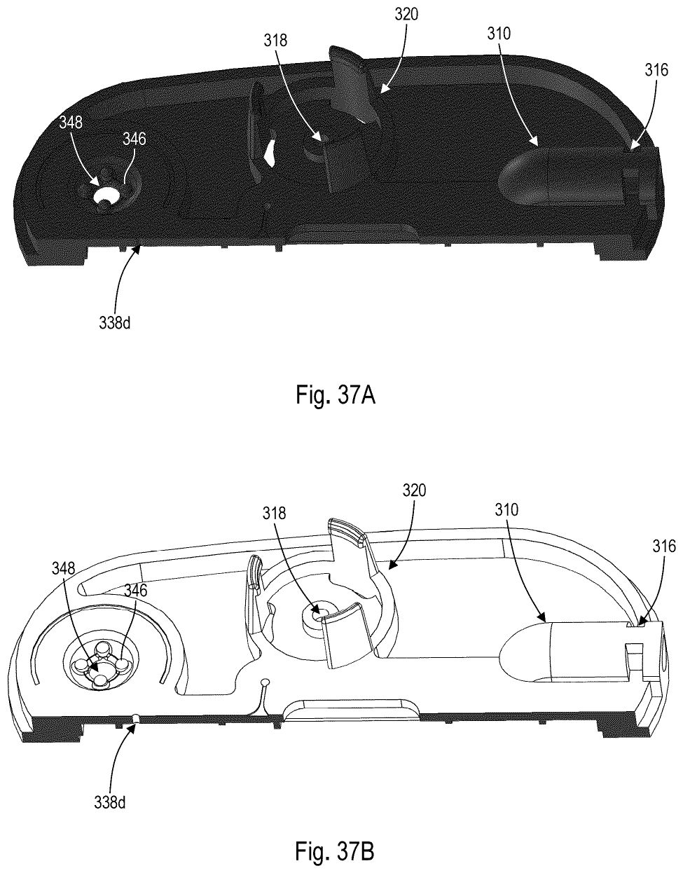

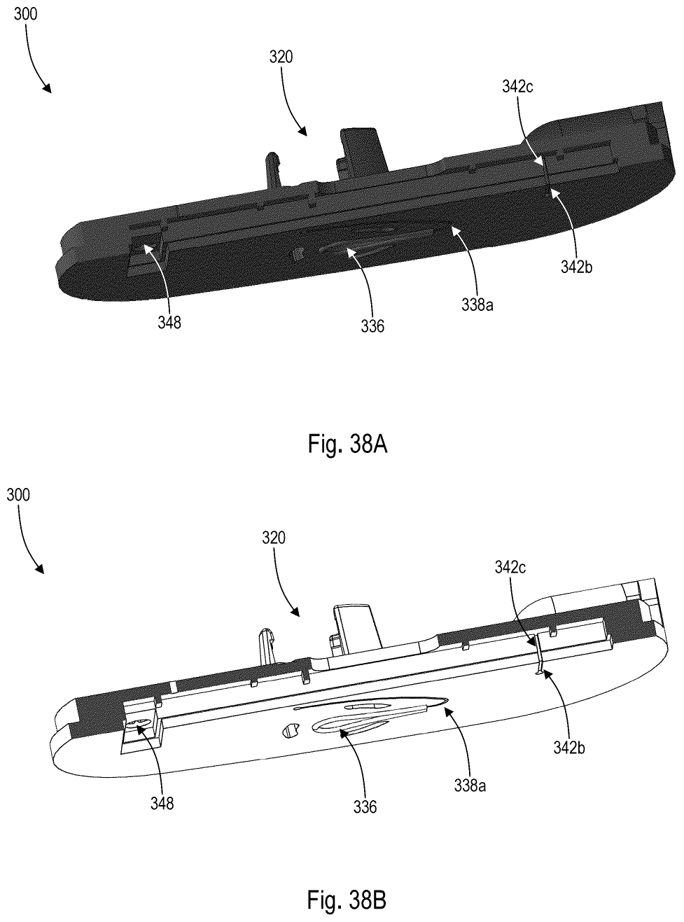

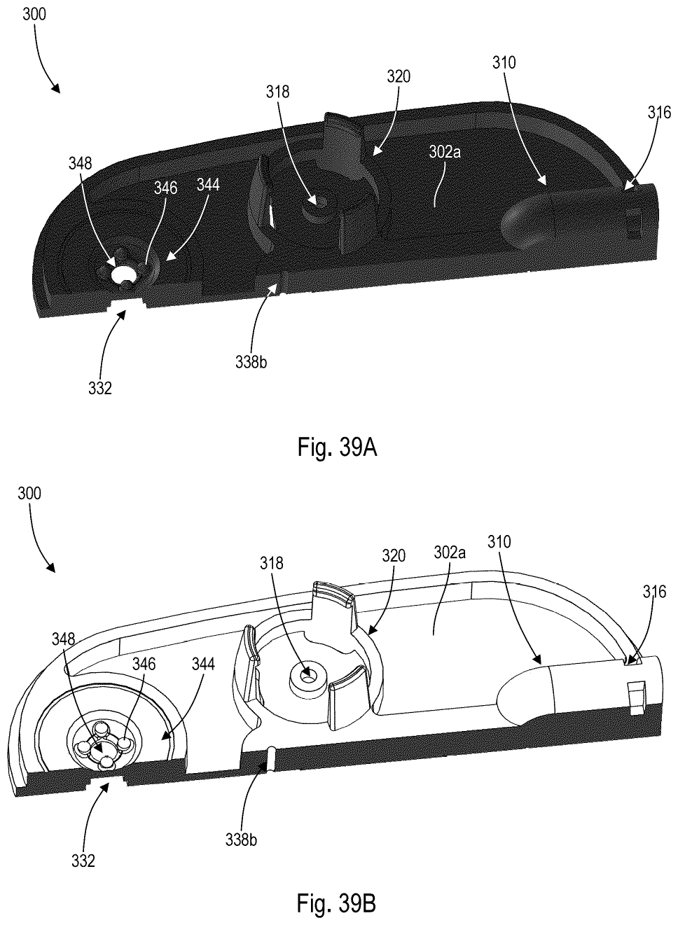

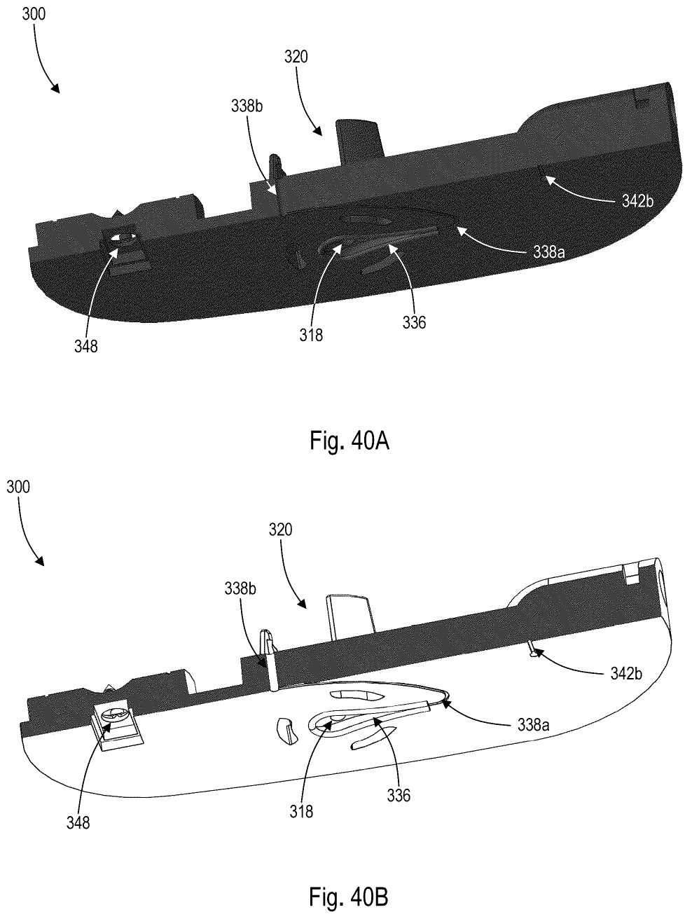

Referring now to FIGS. 23A and 23B-40A and 40B, the base 300 is shown in accordance with an exemplary embodiment. In this embodiment, the base 300 includes a bottom wall 302 with a top surface 302a, an opposed bottom surface 302b, and an outer surface 302c that extends between the top surface 302a and the bottom surface 302b. The top surface 302a and the bottom surface 302b extend perpendicularly to the outer surface 302c. The outer surface 302c extends perpendicular to and vertically between the top surface 302a and the bottom surface 302b. The bottom wall 302 and the side wall 204 of the cover 200 have the same perimeter shape such that when the cover 200 is coupled to the base 300, outer surface 302c of the base 300 and the outer surface 204a of the cover 200 are flush with one another. Furthermore, when the cover 200 is coupled to the base 300, the side wall 204 contacts the top surface 302a of the bottom wall 302.

The base 300 further includes a rim 304 with an outer surface 304a, an opposed inner surface 304b, and a top surface 304c that extends between the outer surface 304a and the inner surface 304b. The top surface 304c extends perpendicularly to and longitudinally between the outer surface 304a and the inner surface 304b. The outer surface 304a and the inner surface 304b extend vertically from and perpendicular to the top surface 302a of the bottom wall 302 such that the outer surface 304a and the inner surface 304b extend between the top surface 302a and the top surface 304c. The rim 304 is contoured such that when the cover 200 is coupled to the base, at least a portion of the side wall 204 contacts at least a portion of the rim 304. More specifically, at least a portion of the inner surface 204b of the side wall 204 contacts at least a portion of the outer surface 304a of the rim 304.

The base 300 further includes a plurality of extensions 306 that extend vertically from and perpendicular to the rim 304. The extensions 306, the bottom wall 302 and the rim 304 define gaps 308. The gaps 308 and therefore the extensions 306, are shaped to accept a locking member 234 such that an extension 306 couples to a locking member 234 via a snap fitting thereby coupling the cover 200 to the base 300.

The base 300 also includes a vacuum pin receptacle 310 that extends vertically from and perpendicular to the top surface 302a of the bottom wall 302. The vacuum pin receptacle 310 includes an opening 312 and a chamber 314 that are each shaped to accept the vacuum pin 400 such that at least a portion of the vacuum pin 400 may be disposed within the vacuum pin receptacle 310. The vacuum pin receptacle 310 also includes a gap 316 that is shaped and dimensioned to accommodate the arms of the U-shaped locking member 224. That is, when the cover 200 is coupled to the base 300, the arms of the U-shaped locking member 224 extend through and are disposed within the gap 316.

The base 300 also includes a needle aperture 318 that is generally circular in shape. The needle aperture 318 extends through the bottom wall 302. Stated another way, the needle aperture 318 extends between the top surface 302a and the bottom surface 302b of the bottom wall 302. As will be discussed in further detail herein, when the cover 200 is coupled to the base 300 and when the cartridge 12 is adhered to a subject, the needle aperture 318 allows the needle 110 of the lancet 100 to extend through the bottom wall 302 to puncture the subject's skin, thereby allowing extraction of a physiological sample from the subject.

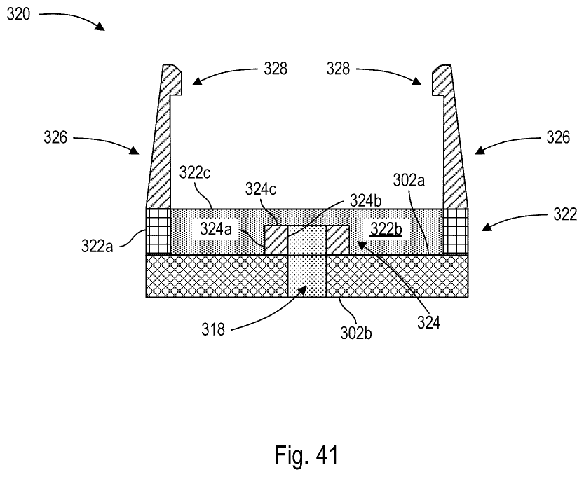

The base 300 further includes a lancet receiving element 320 that is shaped and dimensioned to accept the distal end of the lancet 100. With particular reference to FIG. 41, the lancet receiving element 320 includes an outer circular projection 322 and an inner circular projection 324 with each extending vertically from and perpendicular to the top surface 302a of the bottom wall 302. The outer circular projection 322 includes an outer surface 322a, an opposed inner surface 322b, and a top surface 322c that extends between the outer surface 322a and the inner surface 322b. The top surface 322c extends perpendicular to and longitudinally between the outer surface 322a and the inner surface 322b. The outer surface 322a and the inner surface 322b extend vertically from and perpendicular to the top surface 302a of the bottom wall 302 such that the outer surface 322a and the inner surface 322b extend between the top surface 302a and the top surface 322c. The outer circular projection 322 is shaped to accept the lancet 100.

The inner circular projection 324 is disposed around the needle aperture 318 and includes an outer surface 324a, an opposed inner surface 324b, and a top surface 324c that extends between the outer surface 324a and the inner surface 324b. The top surface 324c extends perpendicular to and longitudinally between the outer surface 324a and the inner surface 324b. The outer surface 324a and the inner surface 324b extend vertically from and perpendicular to the top surface 302a of the bottom wall 302 such that the outer surface 324a and the inner surface 324b extend between the top surface 302a and the top surface 324c. Furthermore, the outer circular projection 322 and the inner circular projection 324 circular projection are concentric with one another. As will be discussed in further detail herein, when the lancet 100 is engaged with the base 300, the top surface 324c of the inner circular projection contacts a portion of the lancet 100 which allows the lancet 100 to release the needle 110 so as to puncture the skin, thereby allowing the extraction of a physiological sample from the subject's skin.

With continued reference to FIGS. 23A and 23B, the base 300 further includes a plurality of locking members 326 (FIG. 41) that extend vertically from and perpendicular to the top surface 322c of the outer circular projection 322. For the sake of clarity, one of the locking members 326 is not shown in FIGS. 23A and 23B. Each locking member 326 includes a hook 328 (FIG. 41) that extends inwardly from a top of a locking member 326 towards the inner circular projection 324. As will be discussed in further detail herein, the hooks 328 of the locking members 326 couple to the lancet 100 to retain the lancet 100 within the base 300. The locking members 326 are equally spaced around the outer circular projection 322 thereby defining a gap between the locking members 326. When the cover 200 is coupled to the base 300, the projection members 226 extend between the locking members 326 in these gaps thereby coupling the cover 200 to the base 300.

The base 300 includes a diagnostic test strip housing 330. The diagnostic test strip housing 330 extends vertically from and perpendicular to the top surface 302a and includes an outer surface 330a an opposed inner surface 330b and a vertical surface 330c. The vertical surface 330c extends perpendicular to and vertically between the inner surface 330b and the bottom surface 302b. The inner surface 330b and vertical surface 330c define an inner volume 332 of the diagnostic test strip housing 330. The diagnostic test strip housing 330 further includes an opening 334 that extends between the outer surface 330a and the inner surface 330b. When the cover 200 is coupled to the base 300 the opening 334 is positioned vertically below the test strip viewing aperture 216.

The base 300 further includes a physiological sample well 336 and a physiological sample channel 338 with a first portion 338a, a second portion 338b, a third portion 338c, and a fourth portion 338d.

The first portion 338a extends from the physiological sample well 336. As will be discussed in further detail herein, the physiological sample channel 338 is a fluidic channel that is configured to carry a physiological sample extracted from a subject. The physiological sample well 336 and the first portion 338a of the physiological sample channel 338 are open with respect to the bottom surface 302b of the bottom wall 302. Stated another way, the physiological sample well 336 and the first portion 338a of the physiological sample channel 338 do not include a bottom surface. The physiological sample well 336 is in open communication with the needle aperture 318. As will be discussed in further detail herein, when drawing a physiological sample, a needle of the lancet 100 extends through the needle aperture 318 and through the physiological sample well 336 to pierce the skin of the subject.

The second portion 338b of the physiological sample channel 338 extends vertically from and perpendicular to the first portion 338a. The second portion 338b extends through the bottom wall 302 and the diagnostic test strip housing 330. That is, the second portion 338b extends between the bottom surface 302b of the bottom wall 302 and the outer surface 330a of the diagnostic test strip housing 330.

The third portion 338c extends longitudinally from and perpendicular to the second portion 338b. The third portion 338c extends along the outer surface 330a of the diagnostic test strip housing 330. The third portion 338c of the physiological sample channel 338 is open with respect to the outer surface 330a of the diagnostic test strip housing 330. Stated another way, the third portion 338c of the physiological sample channel 338 does not include a top surface. The third portion 338c includes a reservoir 340. When the cover 200 is coupled to the base 300, the reservoir 340 is disposed below the sample viewing aperture 214 which allows a user of the dermal patch system 10 to view a drawn physiological sample within the reservoir 340.

The fourth portion 338d of the physiological sample channel 338 extends vertically from and perpendicular to the third portion 338c. The fourth portion 338d extends between outer surface 330a and the inner surface 330b of the diagnostic test strip housing 330 such that the physiological sample channel 338 is in open communication with the inner volume 332.

The base 300 further includes a vacuum channel 342 that is in fluid communication with the chamber 314 of the vacuum pin receptacle 310. A first portion 342a of the vacuum channel 342 extends from the chamber 314 and extends vertically within the base 300. A second portion 342b of the vacuum channel 342 extends longitudinally from and perpendicular to the first portion 342a of the vacuum channel 342 such that the second portion 342b of the vacuum channel 342 extends along the bottom surface 302b of the bottom wall 302. Similar to the physiological sample well 336 and the first portion 338a of the physiological sample channel 338, the second portion 342b of the vacuum channel 342 is open with respect to the bottom surface 302b of the bottom wall 302. The vacuum channel 342 further includes a third portion 342c that extends vertically from and perpendicular to the second portion 342b. The third portion 342c extends vertically along the vertical surface 330c of the diagnostic test strip housing 330. The vacuum channel 342 is in open communication with the inner volume 332 of the diagnostic test strip housing 330. As such, the vacuum channel 342 is in open communication with the physiological sample well 336 via the inner volume 332 and the physiological sample channel 338.

The base 300 further includes a depression 344 and a plurality of piercing elements 346 that extend vertically from the depression 344. The base 300 also includes a buffer aperture 348 that extends between the depression 344 and the inner surface 330b.

As previously discussed herein, the first portion 338a and the third portion 338c of the physiological sample channel 338 are open. As depicted in FIGS. 4A and 4B, the adhesive layer 14 is disposed on the bottom surface 302b of the bottom wall 302 thereby sealing the first portion 338a of the physiological sample channel 338. Furthermore, as depicted in FIGS. 8A and 8B, the film 20 is disposed on the outer surface 330a of the diagnostic test strip housing 330 thereby sealing the third portion 338c of the physiological sample channel 338.

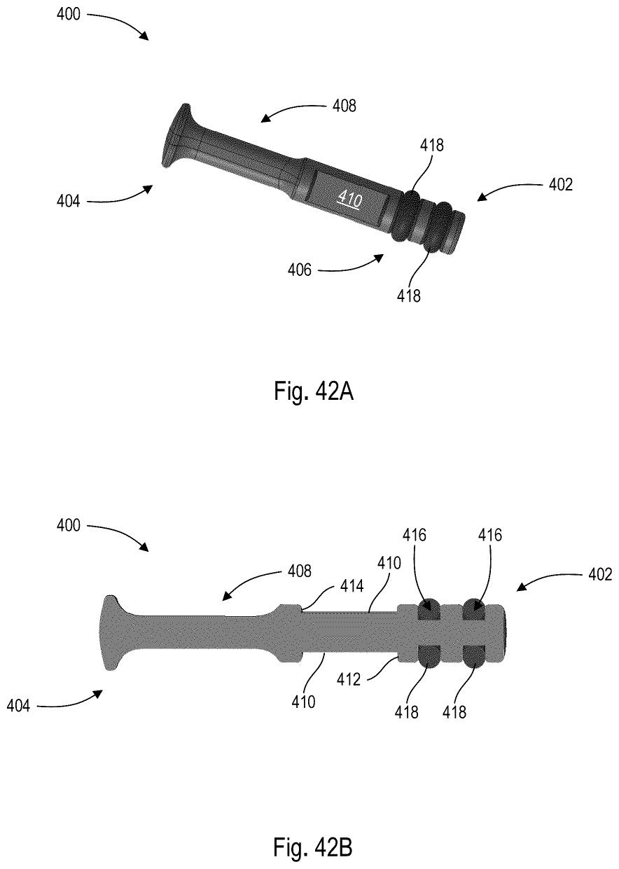

With reference to FIGS. 42A and 42B, the vacuum pin 400 is shown in accordance with an exemplary embodiment. The vacuum pin 400 is generally cylindrical in shape and extends between a proximal end 402 and a distal end 404. The vacuum pin 400 includes a barrel 406 that defines the proximal end 402 and a handle 408 that defines the distal end 404. The vacuum pin 400 also includes a first and second flat surface 410 between the barrel 406 and the handle 408. The flat surfaces 410 extend longitudinally between a first rim 412 and a second rim 414. The flat surfaces 410 extend perpendicular to and longitudinally between the rims 412 and 414.

The barrel 406 includes a first groove 416 and a second groove 416 shaped and dimensioned to accommodate a first and second elastomeric O-ring 418. When the vacuum pin 400 is disposed within the vacuum pin receptacle 310, the elastomeric O-rings 418 contact the inner surface of the chamber 314 such that the vacuum pin 400 creates an airtight seal within the chamber 314. This seal allows for the application of positive or negative pressure as needed.

The vacuum pin 400 may be moved within or completely removed from vacuum pin receptacle 310. When the vacuum pin 400 is transitioned from an undeployed portion (i.e., a position in which the vacuum pin 400 is fully inserted within the chamber 314 of the vacuum pin receptacle 310) to a deployed position (i.e., when the vacuum pin is moved within the vacuum pin receptacle away from the center of the base 300), a volume between the proximal end 402 of the vacuum pin 400 and the chamber 314 increases thereby creating a negative pressure within the chamber 314 which in turn causes the creation of a negative pressure within the physiological sample channel 338 via the vacuum channel 342 and the diagnostic test strip housing 330. Stated another way, when the vacuum pin 400 is moved from the undeployed position to the to the deployed position, the vacuum pin 400 creates a vacuum within the base 300 which draws a physiological sample from the physiological sample well 336 to the diagnostic test strip 18.

As previously discussed herein, the cover 200 includes a U-shaped locking member 224. When the cover 200 is coupled to base 300 the arms of the U-shaped locking member 224 extend through the gap 316 of the vacuum pin receptacle 310. Furthermore, when the vacuum pin 400 is in the undeployed position, the arms of the U-shaped locking member 224 are disposed between the rims 412 and 414. When the vacuum pin 400 is moved to the deployed position, the arms of the U-shaped locking member 224 contact the first rim 412 thereby preventing the vacuum pin 400 from moving further. As previously discussed, moving the vacuum pin 400 to the deployed position creates a vacuum within the base 300. Accordingly, the extent of movement of the vacuum pin 400 permitted by the U-shaped locking member 224 can determine the strength of a vacuum created within the base 300. The rims 412 and 414 are separated by a given distance. In other embodiments of the vacuum pin 400, the rims 412 and 414 are separated by a difference distance. This distance determines the extent by which the vacuum pin 400 can be removed from the vacuum pin receptacle 310 and as such can determine the strength of a vacuum created within the base 300. Hence, increasing or decreasing a distance between rims 412 and 414 increases or decreases the strength of a vacuum that can be created by the vacuum pin 400. The strength or amount of vacuum may also be increased by increasing a length of the vacuum pin receptacle 310, by increasing length of the vacuum pin 400, and/or by increasing the diameter of the vacuum pin receptacle 310.

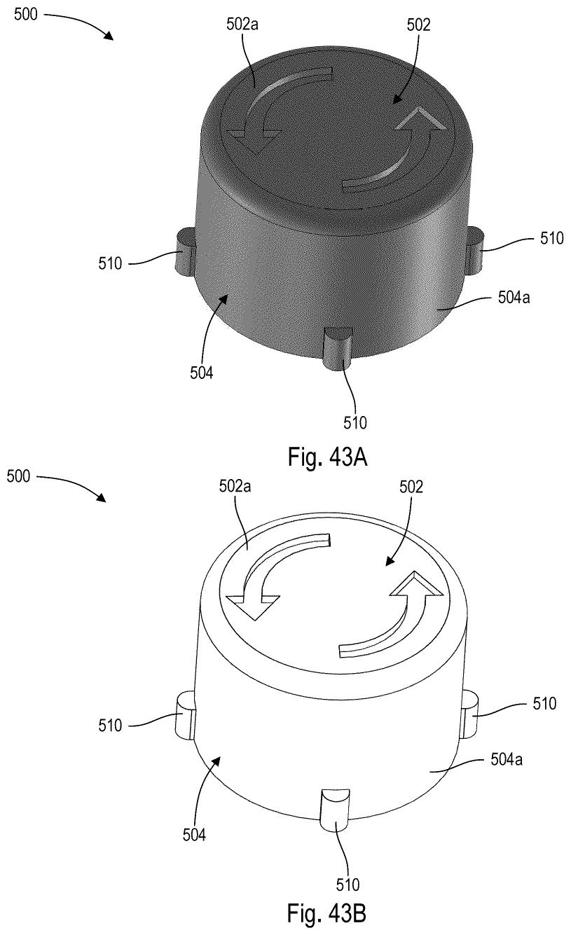

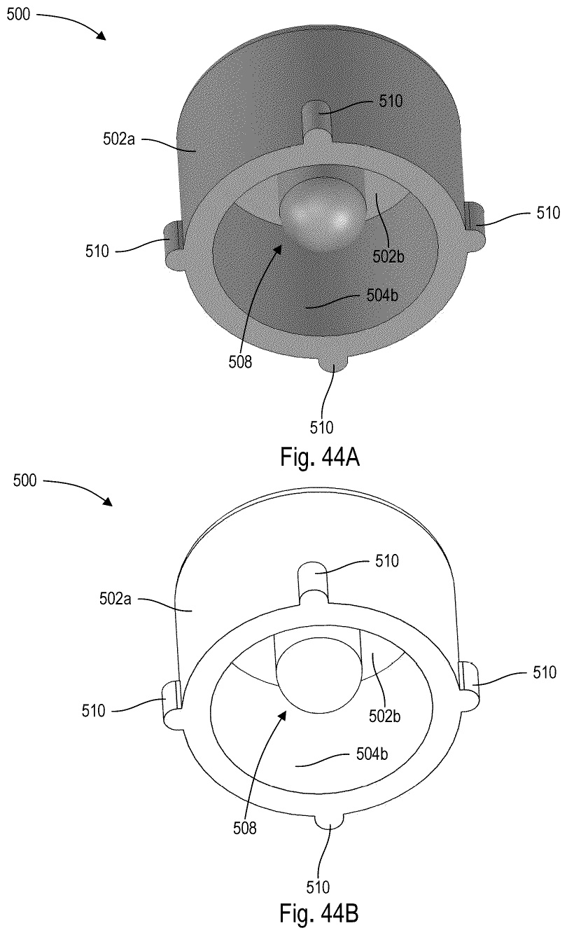

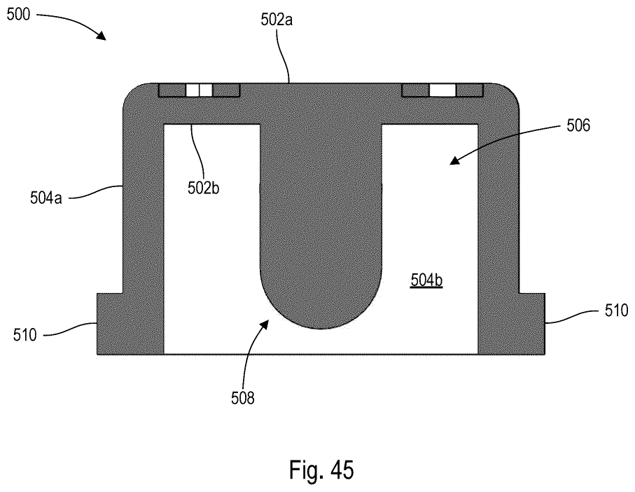

With reference to FIGS. 43A, 43B-45 the button 500 is depicted in accordance with an exemplary embodiment. The button 500 includes a top wall 502 with an outer surface 502a and an opposed inner surface 502b. The button 500 also includes a side wall 504 with an outer surface 504a and an opposed inner surface 504b. The top wall 502 extends longitudinally from and perpendicular to the side wall 504. The side wall 504 extends vertically from and perpendicular to the top wall 502. The top wall 502 and the side wall 504 have generally circular cross sections and are concentric with one another. The inner surface 502a of the top wall 502 and the inner surface 504a of the side wall 504 define an inner volume 506 of the button 500.

The button 500 also includes a cylinder 508 that extends vertically from and perpendicular to the inner surface 502b of the top wall 502. The cylinder 508 is concentric with the top wall 502 and the side wall 504. The button 500 also includes a plurality of protrusions 510 that extend from the outer surface 504a of the side wall 504. The protrusions 510 couple the button 500 to the cover 200.

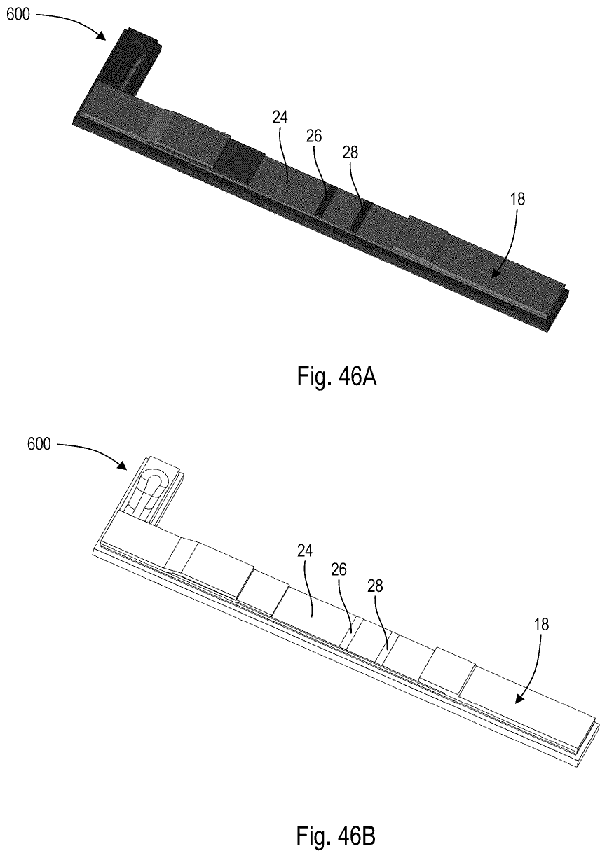

With reference to FIGS. 46A and 46B, the test strip support 600 and the diagnostic test strip 18 is shown in accordance with an exemplary embodiment.

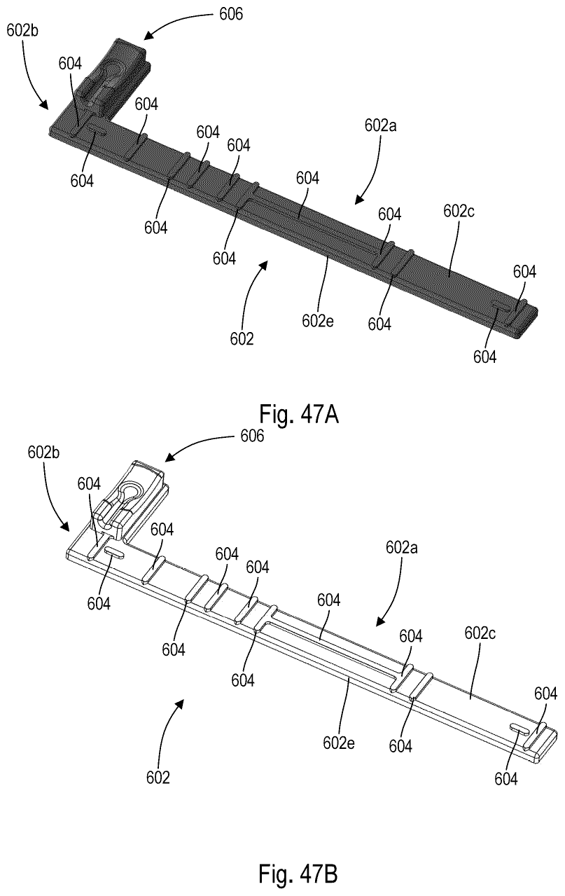

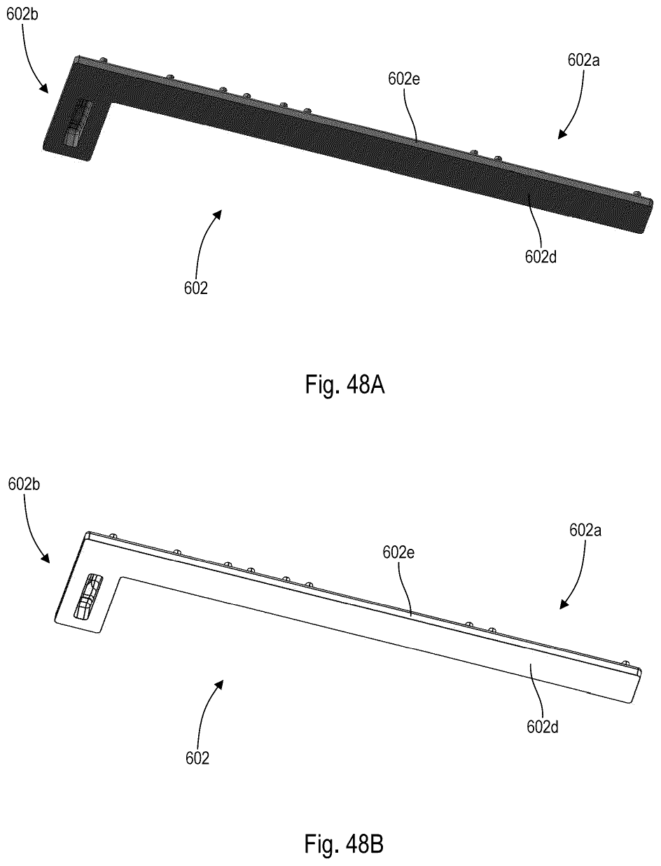

As shown in FIGS. 47A, 47B, 48A, and 48B, the test strip support 600 includes an L-shaped base 602 with a first portion 602a and a second portion 602b that is perpendicular the first portion 602a.

The base 602 includes a top surface 602c, an opposed bottom surface 602d, and a side surface 602e that extends vertically between and perpendicular the top surface 602c and the bottom surface 602d. The side surface 602e gives the base 602 a height substantially that matches a height of the bottom wall 302 of the base 300. As such when the test strip support 600 is coupled to the base 300, the top surface 602c of the base 602 is flush with the top surface 302a of the bottom wall 302 and the bottom surface 602d of the base 602 is flush with the bottom surface 302b of the bottom wall 302.

The first portion 602a of the base 602 is shaped and dimensioned to support the diagnostic test strip 18. Furthermore, the base 602 includes a plurality of protrusions 604 that extend vertically from and perpendicular to the top surface 602c of the base 602. When the diagnostic test strip 18 is disposed on the test strip support 600, the diagnostic test strip 18 rests upon the protrusions 604. The protrusions 604 can prevent the test strip 18 from delaminating (i.e., the protrusions 604 can ensure that layers of the test strip 18 contact one another such that the physiological sample can transfer from one layer to another).

The second portion 602b of the base 602 includes a well 606 that extends vertically from and perpendicular to the top surface 602c of the base 602. When the test strip support 600 is coupled to the base 300, the well 606 extends into the inner volume 332 of the diagnostic test strip housing 330 and is positioned vertically below the buffer aperture 348. Furthermore, the well 606 is shaped and dimensioned to accept a processing fluid from the processing fluid pack 16 via the buffer aperture 348. A bottom surface of the well 606 is angled toward the first portion 602a of the base 602 and therefore directs a received fluid towards the diagnostic test strip 18 when the diagnostic test strip 18 is disposed on the first portion 602a of the base 602.

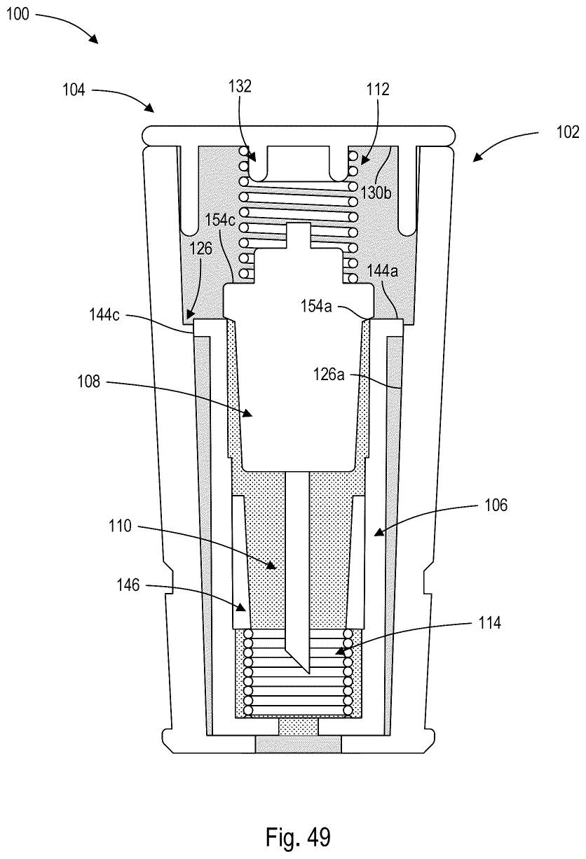

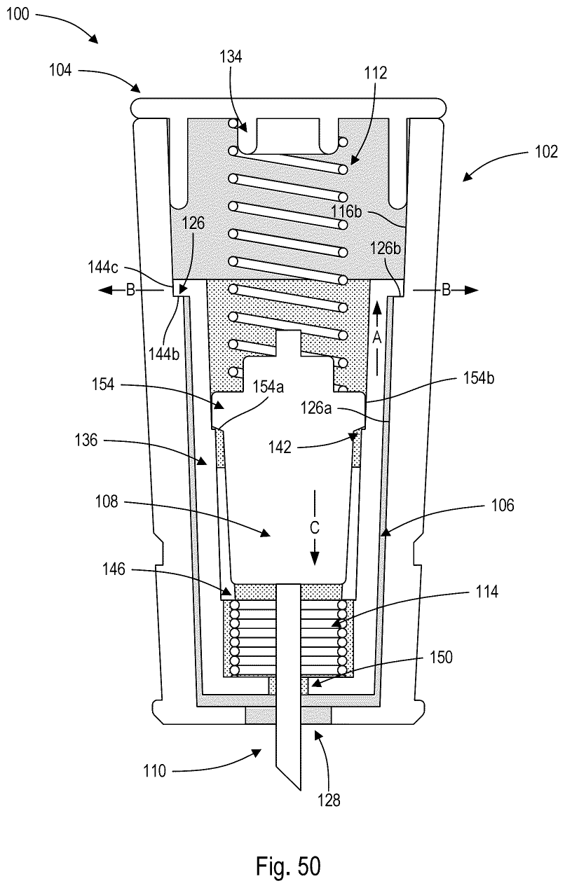

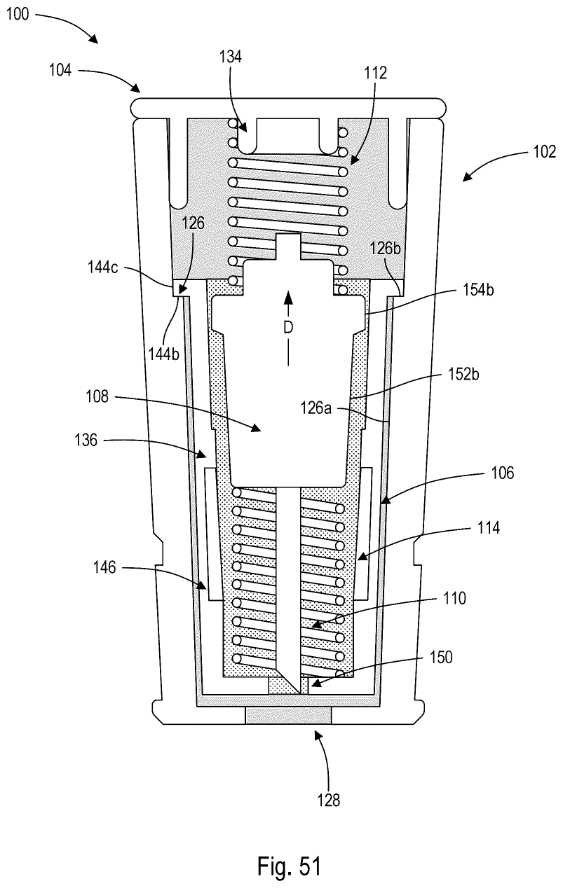

As depicted in FIGS. 49-51, the lancet 100 is moveable between a first position (also referred to as an “undeployed position”) (FIG. 49), a second position (also referred to as a “deployed position”) (FIG. 50), and a third position (also referred to as a “retracted position”) (FIG. 51).

In the undeployed position (before the lancet 100 is inserted into the cartridge 12; FIG. 49) the injection spring 112 and the retraction spring 114 are in a compressed state. In the compressed state, the retraction spring 114 extends vertically between the bottom wall 138 and a proximal end of the locking members 146. More specifically, a distal end of the retraction spring 114 contacts a lower surface of the proximal end of the locking members 146 and a proximal end of the retraction spring 114 contacts the inner surface 138b of the bottom wall 138.

When in the undeployed position the outer surface 144c contacts the inner surface 126a of the columns 126 which compresses the side wall 136 inwardly. Furthermore, the bottom surface 154a of the second cylinder 154 contacts and rests upon the top surfaces 144a of the ledges 144 such that the ledges 144 supports the needle frame 108 in the undeployed position. In this position, the injection spring 112 is prevented from decompressing (due to the second cylinder 154 resting upon the ledges 144) and the needle 110 is disposed completely within the inner volume 140 of the inner sleeve 106.

When the lancet 100 is inserted into the cartridge 12, the engagement of the lancet with the cartridge 12 causes the lancet 100 to automatically move from the undeployed position to the deployed position.

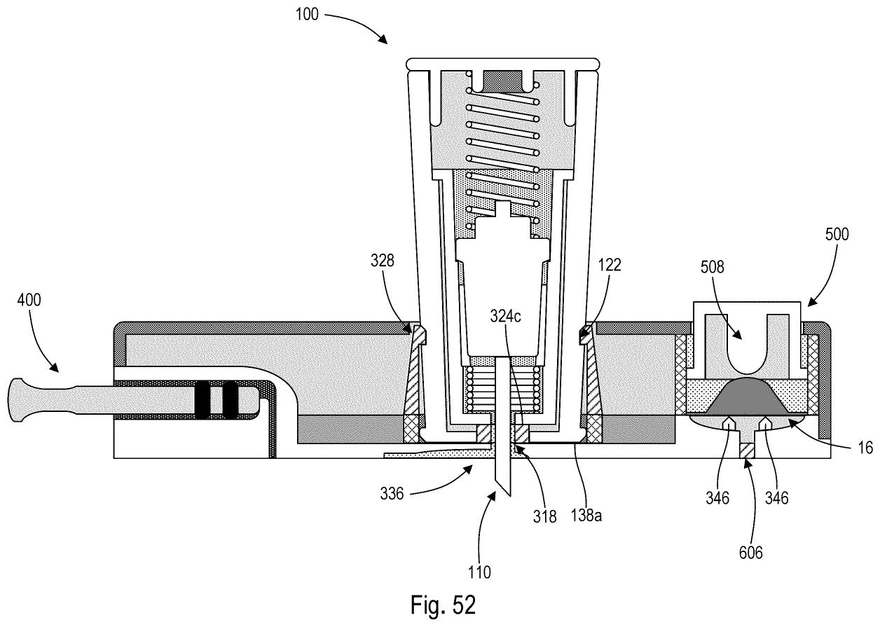

When the lancet 100 is coupled to the base 300 (FIGS. 52-55), the inner circular projection 324 extends through the aperture 128 to contact the bottom wall 138. Specifically, the top surface 324c of the inner circular projection 324 contacts the outer surface 138a of the bottom wall 138 which forces the inner sleeve 106 to move vertically upward in the direction of arrow A (FIG. 50) within the housing 102. This vertical movement causes the ledges 144 to extend vertically above the top surfaces 126b of the columns 126. Moving beyond the top surfaces 126b of the columns 126 allows the side wall 136 to decompress and expand in the direction of arrow B (FIG. 50) and extend toward the inner surface 116b of the side wall 116. In this position, the bottom surface 144b of the ledges 144 rest upon the top surfaces 126b of the columns 126 and the outer surfaces 144c of the ledges 144 contacts the inner surface 116b of the side wall 116. Furthermore, when the lancet 100 is inserted into the cartridge 12, the hooks 328 of the locking members 326 are disposed within and coupled to the notch 122 via a snap fit.

The expansion of the side wall 136 causes the inner volume 140 of the inner sleeve 106 to have a larger width relative to when the inner sleeve 106 is in the undeployed position such that at least a portion of the side wall 136 has a larger width than the second cylinder 154 (the widest portion of the needle frame 108 which allows the needle frame 108 move vertically downward in the direction of arrow C) (FIG. 50).

Furthermore, the injection spring 112 also causes the needle frame 108 to move in the direction of arrow C as the ledges 144 no longer prevent the injection spring 112 from expanding. The force applied by the injection spring 112 causes the needle frame 108 (and therefore the needle 110) to travel with a force that is sufficient to cause the needle 110 to puncture the skin of a subject wearing the dermal patch system 10. Stated another way, the injection spring 112 causes the needle 110 to extend through the aperture 150 of the inner sleeve 106, through the aperture 128 of the housing 102, and through the needle aperture 318 of the base 300 to puncture the skin of a subject. In the deployed position, the bottom surface 154a of the second cylinder 154 rests upon the columns 142 and at least a portion of the outer surface 154b of the second cylinder 154 contacts the inner surface 136b of the side wall 136.

While moving in the direction of arrow C, the outer surface 152b contacts the locking members 146 which causes a proximal portion of locking members 146 that is aligned with an opening 148 to extend into the opening. In this position, the locking members 146 no longer contact the retraction spring 114 thereby allowing the retraction spring 114 to decompress and expand. When decompressed, the retraction spring 114 contacts the outer surface 152b of the first cylinder 152 which causes needle frame 108 to also move in the direction of arrow D (FIG. 51). That is after moving to the deployed position, the retraction spring 114 causes the needle 110 to retract back into the inner volume 140 of the inner sleeve 106 via the aperture 150 of the base 300 and the apertures 128 and 150 of the lancet 100. After penetrating the skin of a subject, the retraction spring 114 causes the needle 110 to automatically retract back into the housing of the lancet 100 thereby placing the lancet 100 in the retraced position.

As previously discussed herein, the lancet includes a mechanism can be transition the lancet between a locked state and a released state. In various embodiments, this mechanism includes the columns 126, the ledges 144, and the locking members 146. An upper locking portion of the mechanism refers to the columns 126 and the ledges 144 while a locker locking portion refers to the locking members 146 as the columns 126 and the ledges 144 can be positioned vertically above the locking members 146. The term upper interference portion refers to the top surface 144a of the ledges 144 as this surface interferes with the needle frame's 108 ability to transition to the deployed position when the mechanism is in the locked state. As used herein, a lower interference member refers to the columns 142 as the columns 142 interfere with the needle frame's 108 ability to further extend beyond a desired position.

In use, after affixing the cartridge 12 to the skin of the subject, a user of the dermal patch system 10 inserts the lancet into the cartridge 12 which causes the needle 110 to move to the deployed position and puncture the subject's skin and draw a physiological sample. After the needle 110 retracts into the lancet 100, the drawn physiological sample pools within the physiological sample well 336 of the base 300.

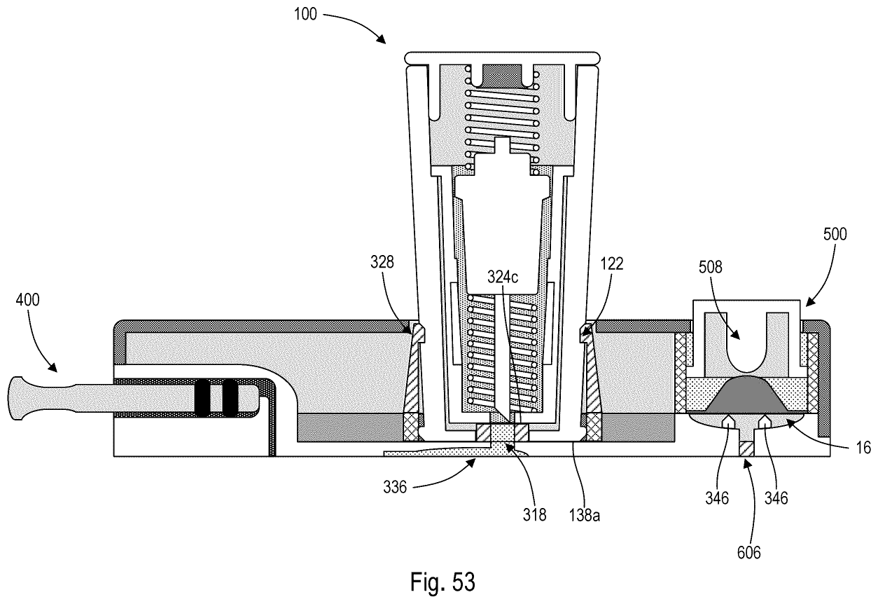

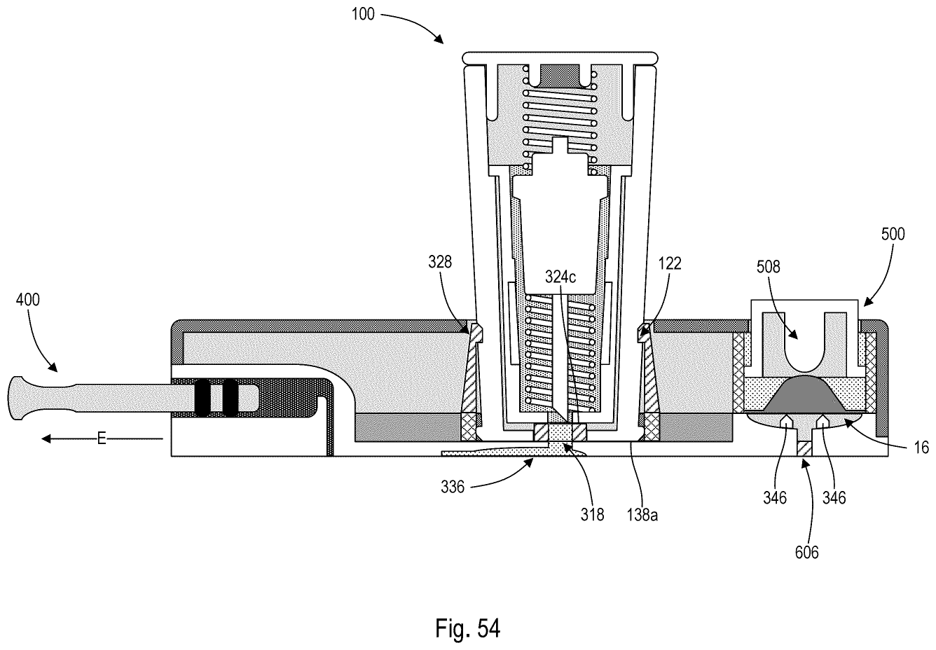

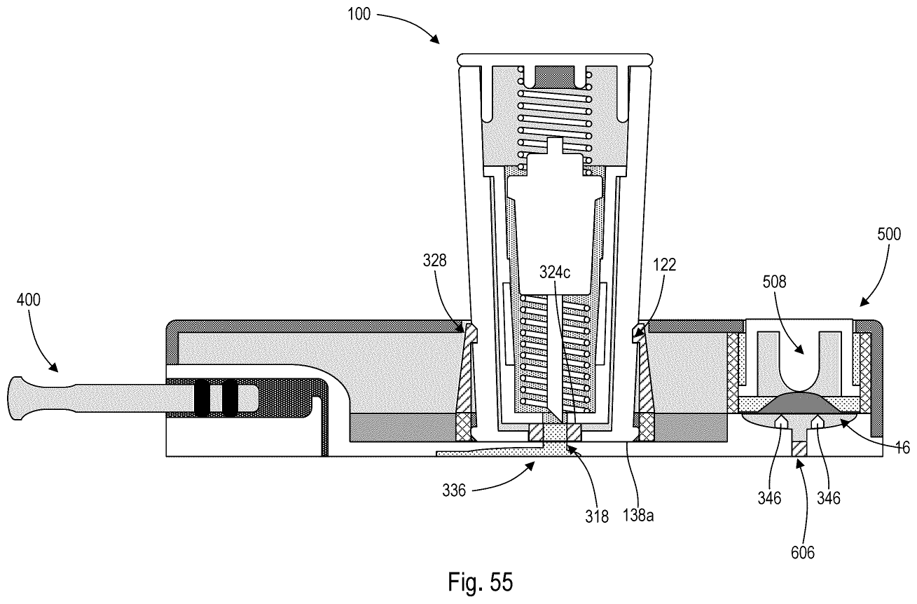

As depicted in FIGS. 52-55, the button 500 is moveable between a first position (also referred to as a “locked” position) (FIGS. 52 and 53), a second position (also referred to as an “unlocked” position), and a third position (also referred to as a “deployed” position) (FIGS. 54 and 55).

In the locked position, the protrusions 510 are disposed between and within the horizontal grooves 232 of the button guide 228. In this position, the horizontal grooves 232 prevent a user of the dermal patch system 10 from moving the button 500 into the deployed position. A user of the dermal patch system 10 can rotate the button 500 to the unlocked position. In the unlocked position the protrusions 510 are aligned with the vertical grooves 230 of the button guide 228 and the horizontal grooves 232 do not prevent a user from moving the button 500 into the deployed position. That is, in the unlocked position, a user of the dermal patch system 10 may move the button 500 into the deployed position by pressing the button 500. When moving to the deployed position, the protrusions 510 travel downward within the vertical grooves 230.

Moving the button 500 to the deployed position places at least a portion of the processing fluid pack 16 within the inner volume 506 and causes the cylinder 508 to compress the processing fluid pack 16 into the piercing elements 346 which causes the processing fluid pack 16 to rupture and release the stored processing fluid. The released processing fluid travels to the diagnostic test strip 18 via the buffer aperture 348 and the well 606. The well 606 can slow the flow of the processing fluid from the processing fluid pack 16 and allows the processing fluid to be more easily wicked by the diagnostic test strip 18 which may mimic the rate at which a diagnostic test strip outside of the dermal patch system 10.

A user of the dermal patch system 10 may move the button 500 to the deployed position thereby releasing the processing fluid before or after drawing the physiological sample.

When the physiological sample is within the physiological sample well 336 and before rupturing the processing fluid pack 16, a user of the dermal patch system 10 can pull the vacuum pin 400 in the direction of arrow E (FIG. 55) to move the vacuum pin 400 from a first position (also referred to as an “undeployed” position) (FIGS. 52-54) to a second position (FIG. 55) (also referred to as an “deployed” position). Moving the vacuum pin 400 to the deployed position creates a vacuum within the vacuum channel 342 which causes the drawn physiological sample to travel to the diagnostic test strip 18 via the physiological sample channel 338. In some such embodiments, the flow of the drawn physiological sample can be aided by capillary action, gravity and wicking action. As previously discussed herein, en route to the diagnostic test strip 18, the drawn physiological sample passes through the reservoir 340. The user may verify the drawn physiological sample is traveling to the diagnostic test strip 18 by verifying at least a portion of the drawn physiological sample is within the reservoir 340 via the sample viewing aperture 214.

After pulling the vacuum pin 400, the user pushes the button 500 which releases the processing fluid and causes the processing fluid to mix and interact with the physiological sample to form a processed physiological sample. The processed physiological sample is washed across the diagnostic test strip 18. The processed physiological sample can be configured to facilitate the detection of a target biomarker of interest, if any, in the processed physiological sample via the diagnostic test strip 18. By way of example, when the physiological sample is the blood, the processing fluid can include an anti-coagulant to prevent coagulation of the drawn sample prior to its analysis. In some cases, the processing fluid can include a lysing agent for lysing cells present in a drawn sample, e.g., to allow analysis of genetic materials, e.g., DNA and/or RNA segments, within the cell.

The diagnostic test strip 18 can include, but is not limited to, a lateral flow assay, a Bio-marker sensing chip, and Iso-thermal amplification technology.

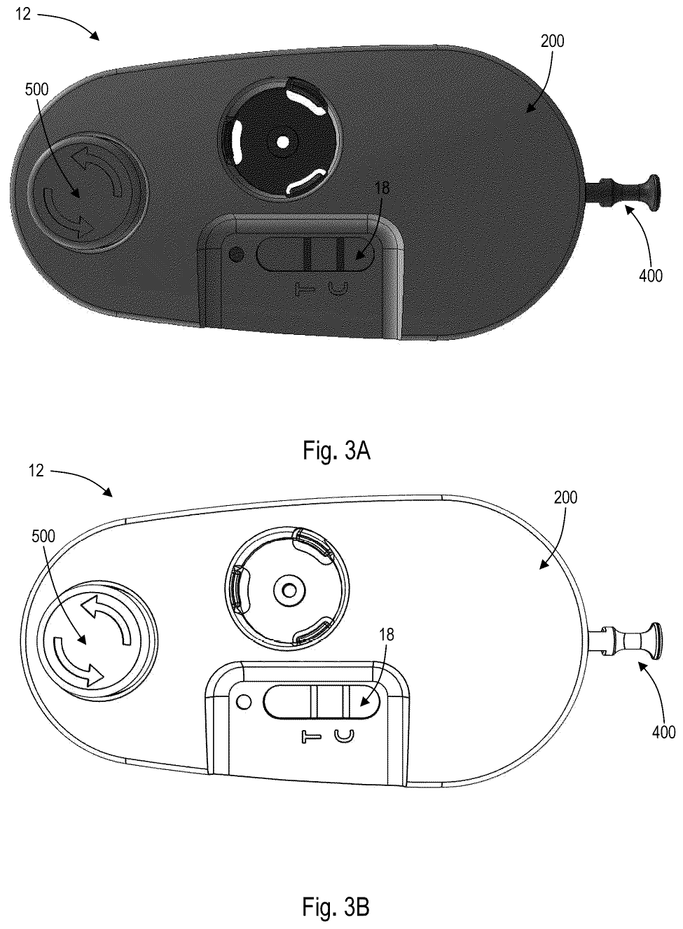

In an embodiment wherein the diagnostic test strip 18 is a lateral flow assay (FIGS. 3A and 3B), the diagnostic test strip 18 includes a nitrocellulose membrane 24 with a line of test capture antibodies 26 (FIGS. 46A and 46B) specific to a desired biomarker and a line of control capture antibodies 28 (FIGS. 46A and 46B). When the diagnostic test strip 18 is disposed within the cartridge 12, the line of test capture antibodies 26 is aligned with test result indicator 218 and the line of control capture antibodies 28 is aligned with the control indicator 220 which allows a user to visually determine if a biomarker (e.g., HbAlc, Cardiac Troponin, CBC, Creatinine, Infectious diseases, etc.) is present in the drawn physiological sample by viewing the diagnostic test strip 18 via the test strip viewing aperture 216.

In some embodiments, after the diagnostic test strip 18 includes the processed physiological sample, the user can remove the dermal patch system 10 from the skin of the subject and can send the dermal patch system to a medical professional. The medical professional can then view the diagnostic test strip 18 to determine if a biomarker is present in the drawn physiological sample.

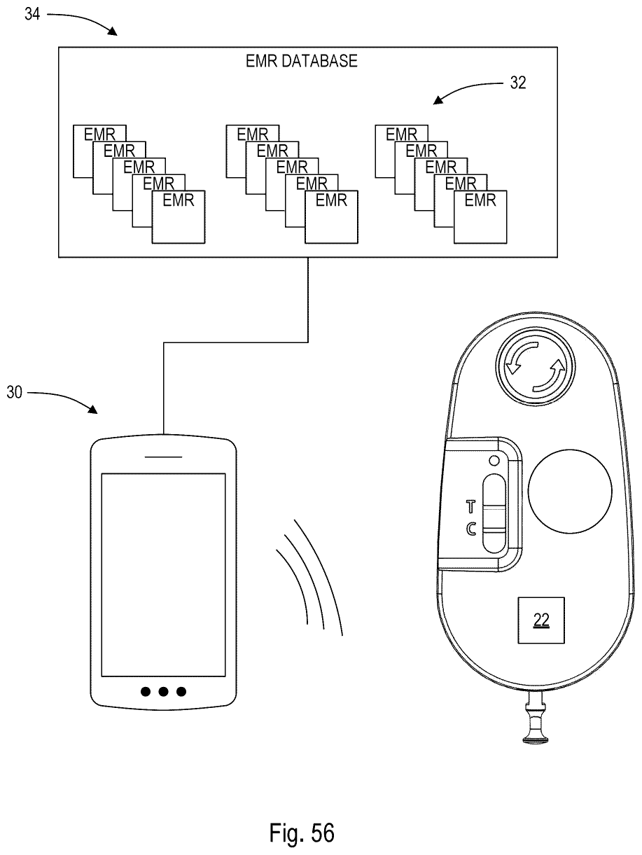

With reference to FIG. 56, in some embodiments wherein the dermal patch system 10 includes the QR code 22, a user of a computer system 30 may scan the QR code 22 to determine a test to perform on the stored physiological sample and/or to view and/or update an EMR 32 that is associated with the QR code 22. In these embodiments, the EMR 32 is stored in an EMR database 34 that is in communication with the computer system 30. Furthermore, the QR code 22 may be employed to preserve the chain of custody of the dermal patch system 10.

In these embodiments, the computer system 30 may include an application that provides access to the EMR database 34 via a network connection and allows a user to photograph of scan the QR code 22. As shown in FIG. 56, the EMR database 34 includes a plurality of EMRs 32 each of which is associated with an individual subject. The application causes the computer system 30 to scan or retrieve an image of the QR code 22, analyze the QR code 22 and associate the QR code 22 with an EMR 32. In some embodiments, the computer system 30 may then update the associated EMR 32 to indicate a diagnostic test has been run on the drawn physiological sample. The computer system 30 may automatically update the EMR 32 automatically or based on a user input. In some embodiments, after associating an EMR 32 with the QR code 22, the computer system 30 may analyze information within the EMR 32 to determine a diagnostic test that associated with the diagnostic test strip 18 and in some embodiments, the computer system 30 may utilize image recognition software to determine a result of the diagnostic test by determining the presence or absence of a test line on the diagnostic test strip 18 in an in an image captured by the computer system 30. In these embodiments, the computer system 30 may automatically update the associated EMR 32 to indicate the determined result.

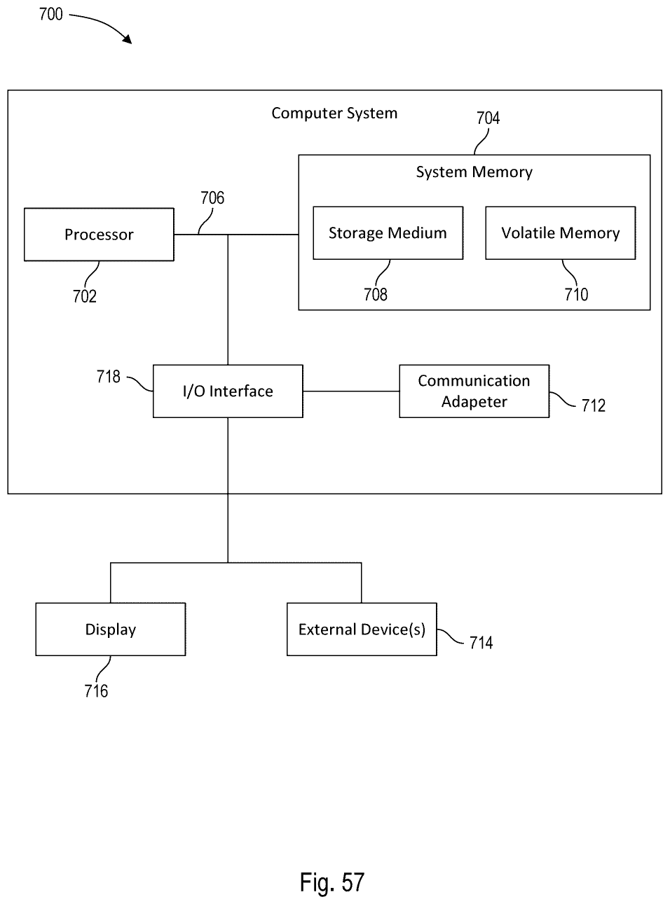

Referring now to FIG. 57, a computer system 700 is shown in accordance with an exemplary embodiment. The computer system 700 may serve as any computer system disclosed herein (e.g., the computer system 30). As used herein a computer system (or device) is any system/device capable of receiving, processing, and/or sending data. Computer systems include, but are not limited to, microprocessor-based systems, personal computers, servers, hand-held computing devices, tablets, smartphones, multiprocessor-based systems, mainframe computer systems, virtual reality (“VR”) headsets and the like.

As shown in FIG. 57, the computer system 700 includes one or more processors or processing units 702, a system memory 704, and a bus 706 that couples the various components of the computer system 700 including the system memory 704 to the processor 702. The system memory 704 includes a computer readable storage medium 708 and volatile memory 710 (e.g., Random Access Memory, cache, etc.). As used herein, a computer readable storage medium includes any media that is capable of storing computer readable; program instructions and is accessible by a processor. The computer readable storage medium 708 includes non-volatile and non-transitory storage media (e.g., flash memory, read only memory (ROM), hard disk drives, etc.). Computer program instructions as described herein include program modules (e.g., routines, programs, objects, components, logic, data structures, etc.) that are executable by a processor. Furthermore, computer readable program instructions, when executed by a processor, can direct a computer system to function in a particular manner such that a computer readable storage medium comprises an article of manufacture. Specifically, the computer readable program instructions when executed by a processor can create a means for carrying out at least a portion of the steps of the methods disclosed herein.

The bus 706 may be one or more of any type of bus structure capable of transmitting data between components of the computer system 700 (e.g., a memory bus, a memory controller, a peripheral bus, an accelerated graphics port, etc.).

The computer system 700 may further include a communication adapter 712 which allows the computer system 700 to communicate with one or more other computer systems/devices via one or more communication protocols (e.g., Wi-Fi, BTLE, etc.) and in some embodiments may allow the computer system 700 to communicate with one or more other computer systems/devices over one or more networks (e.g., a local area network (LAN), a wide area network (WAN), a public network (the Internet), etc.).

In some embodiments, the computer system 700 may be connected to one or more external devices 714 and a display 716. As used herein, an external device includes any device that allows a user to interact with a computer system (e.g., mouse, keyboard, touch screen, etc.). An external device 714 and the display 716 may be in communication with the processor 702 and the system memory 704 via an Input/Output (I/O) interface 718.

The display 716 may display a graphical user interface (GUI) that may include a plurality of selectable icons and/or editable fields. A user may use an external device 714 (e.g., a mouse) to select one or more icons and/or edit one or more editable fields. Selecting an icon and/or editing a field may cause the processor 702 to execute computer readable program instructions stored in the computer readable storage medium 708. In one example, a user may use an external device 714 to interact with the computer system 700 and cause the processor 702 to execute computer readable program instructions relating to at least a portion of the steps of the methods disclosed herein.

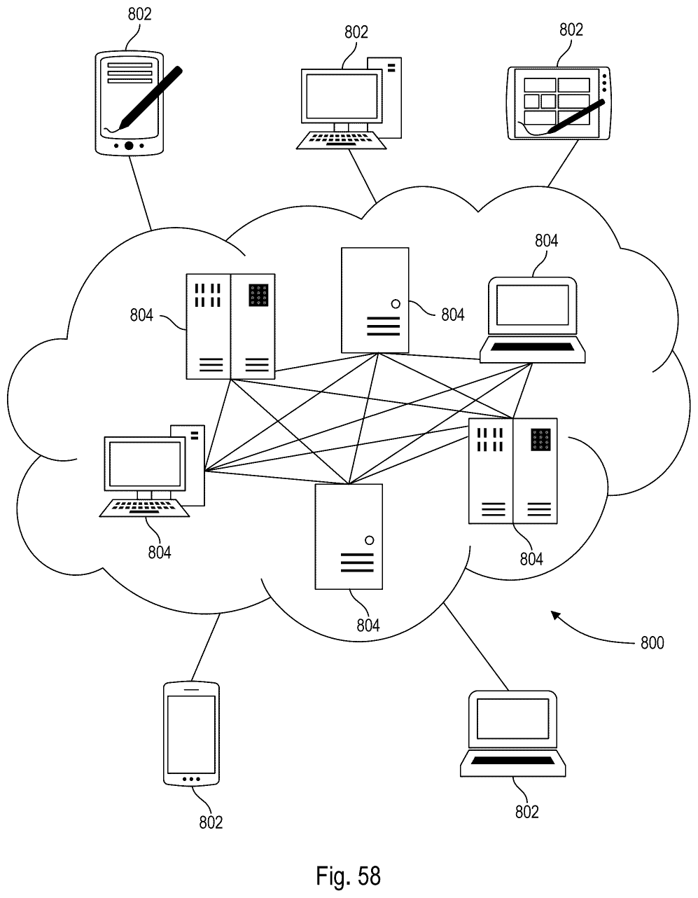

Referring now to FIG. 58, a cloud computing environment 800 is depicted in accordance with an exemplary embodiment. The cloud computing environment 800 is connected to one or more user computer systems 802 and provides access to shared computer resources (e.g., storage, memory, applications, virtual machines, etc.) to the user computer systems 802. As depicted in FIG. 58, the cloud computing environment includes one or more interconnected nodes 804. Each node 804 may be a computer system or device local processing and storage capabilities. The nodes 804 may be grouped and in communication with one another via one or more networks. This allows the cloud computing environment 800 to offer software services to the one or more computer services to the one or more user computer systems 802 and as such, a user computer system 802 does not need to maintain resources locally.

In one embodiment, a node 804 includes computer readable program instructions for carrying out various steps of various methods disclosed herein. In these embodiments, a user of a user computer system 802 that is connected to the cloud computing environment may cause a node 804 to execute the computer readable program instructions to carry out various steps of various methods disclosed herein.

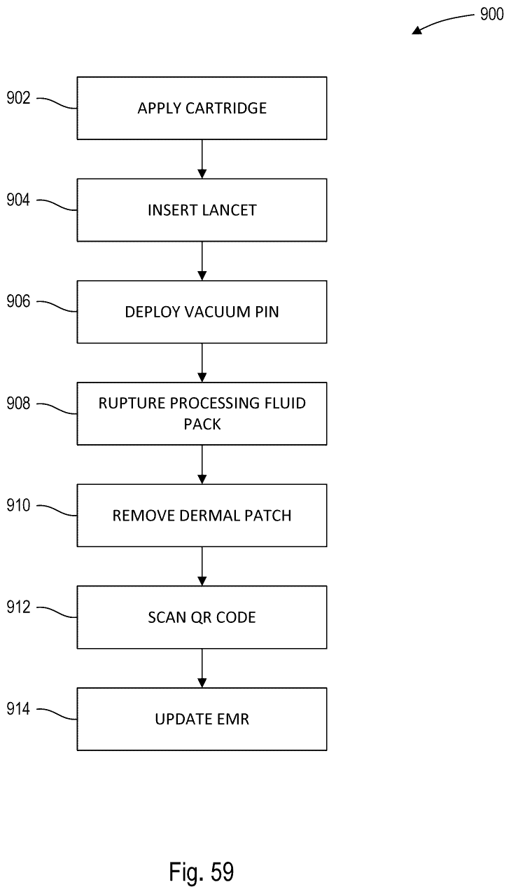

Referring now to FIG. 59, a method 900 for running a diagnostic test on a drawn physiological sample is shown in accordance with an exemplary embodiment.

At 902, a user (e.g., a medical professional, a subject, etc.) applies the cartridge 12 to the skin of the subject via the adhesive layer 14 at a suitable location (e.g., on a leg, arm, etc.) as previously discussed herein.

At 904, the user inserts the lancet 100 into the cartridge 12 thereby causing the needle 110 of the lancet 100 to draw a physiological sample (e.g., a blood sample, a sample of interstitial fluid, etc.) from the subject as previously discussed herein.

At 906, the user moves the vacuum pin 400 from the undeployed position to the deployed position to draw the drawn physiological sample to the diagnostic test strip 18 as previously discussed herein.

At 908, the user ruptures the processing fluid pack 16 by moving the button 500 from the locked position to the deployed position, wherein the drawn physiological sample interacts with the released processing fluid to form a processed physiological sample as previously discussed herein

At 910, the user removes the dermal patch system 10 from the skin of the subject and determines the presence or absence of a biomarker in the drawn physiological sample by viewing the test strip as previously discussed herein.

At 912, a user of the computer system 30 scans the QR code 22 and photographs the diagnostic test strip 18 as previously discussed herein.

At 914, the computer system 30 or another computer system that is in communication with the computer system 30 associates the QR code 22 with an EMR 32 and updates the associated EMR 32 to indicate a diagnostic test has been performed on the drawn physiological sample. In one embodiment, the computer system 30 automatically updates the associated EMR 32. In another embodiment, the user of the computer system 30 updates the associated EMR 32. Furthermore, at 914 the user of the computer system 30 photographs the diagnostic test strip 18 and the computer system 30 associates the photograph with the EMR 32 in the EMR database 34. In one embodiment, the computer system 30 or another computer system connected to the EMR database 34 analyzes the photograph to determine the result of the diagnostic test (i.e., the presence or absence of a biomarker in the drawn physiological sample) and updates the associated EMR 32 to include the determined result.

As previously discussed, some of the steps of the various methods disclosed herein may be implemented by way of computer readable instructions, encoded or embedded on computer readable storage medium (which excludes transitory medium), which, when executed by a processor(s), cause the processor(s) to carry out various steps of the methods of the present disclosure.

While various embodiments have been illustrated and described in detail in the drawings and foregoing description, such illustration and description are to be considered illustrative or exemplary and not restrictive; embodiments of the present disclosure are not limited to the disclosed embodiments. Other variations to the disclosed embodiments can be understood and effected by those skilled in the art in practicing embodiments of the present disclosure, from a study of the drawings, the disclosure, and the appended claims.

In the claims, the word “comprising” does not exclude other elements or steps, and the indefinite article “a” or “an” does not exclude a plurality. A single processor or other processing unit may fulfill the functions of several items recited in the claims. The mere fact that certain measures are recited in mutually different dependent claims does not indicate that a combination of these measured cannot be used to advantage. Any reference signs in the claims should not be construed as limiting the scope.

What is claimed is:

1. A method for analyzing a physiological sample, comprising:
affixing a cartridge to the skin of a subject,
inserting a lancet with a needle into the cartridge,
wherein inserting the lancet into the cartridge deploys the needle to draw a physiological sample from the subject,
moving a vacuum pin to create a vacuum within the cartridge which draws the drawn physiological sample to the diagnostic test strip, and
rupturing a processing fluid pack of the cartridge to release a processing fluid to the diagnostic test strip.

2. The method of claim 1, further comprising:
moving a button of the cartridge to a deployed position to compress the processing fluid pack into a piercing element of the cartridge thereby rupturing the processing fluid pack.

3. The method of claim 1, wherein inserting the lancet into the cartridge causes the lancet to automatically deploy the needle.

4. A method for obtaining a physiological sample, comprising:
affixing a cartridge to the skin of a subject; and
inserting a lancet with a needle into the cartridge, and thereby causing the cartridge to deploy the needle to draw a physiological sample from the subject.

5. The method of claim 4, wherein the inserting of the lancet into the cartridge causes the cartridge to automatically retract the needle into a housing of the lancet after the deployment of the needle.

6. The method of claim 4, further comprising moving a vacuum pin to create a vacuum within the cartridge and thereby cause the physiological sample to flow to a diagnostic test strip.

7. The method of claim 6, wherein a strength of the vacuum varies based on a distance the moving moves the vacuum pin.

8. The method of claim 4, wherein the drawing of the physiological sample from the subject causes the physiological sample to flow to a physiological sample well in the cartridge.

9. The method of claim 8, further comprising moving a vacuum pin to create a vacuum within the cartridge and thereby cause the physiological sample to flow from the physiological sample well to a diagnostic test strip.
